(19) United States
(12) Patent Application Publication
(10) Pub. No.: US 2013/0224818 A1
(43) Pub. Date: Aug. 29, 2013

(54) METHOD FOR PREPARING A HYDROCARBON

(71) Applicant: Shell Oil Company, (US)

(72) Inventors: Thomas Paul HOWARD, Devon (GB); Sabine MIDDELHAUFE, Devon (GB); Dagmara KOLAK, Exeter (GB); Stephen J. AVES, Exeter, Devon (GB); John LOVE, Grantchester (GB); David PARKER, Chester (GB); George Robert LEE, Chester Cheshire (GB)

(73) Assignee: SHELL OIL COMPANY, Houston, TX (US)

(21) Appl. No.: 13/774,647

(22) Filed: Feb. 22, 2013

(30) Foreign Application Priority Data
Feb. 24, 2012 (EP) 12156914.9
May 9, 2012 (EP) 12167393.3

(51) Int. Cl. C12P 5/02

(52) U.S. Cl. CPC C12P 5/02
USPC 435/167; 435/252.33; 435/254.21; 435/320.1

(57) ABSTRACT

A method for preparing a hydrocarbon comprising contacting a fatty acid substrate with at least one fatty acid reductase and at least one fatty aldehyde synthetase and at least one fatty acyl transferase, wherein the fatty acid substrate is a fatty acid, a fatty acyl-ACP, or a fatty acyl-CoA or a mixture of any of these, to obtain a fatty aldehyde; and contacting the fatty aldehyde with at least one aldehyde decarbonylase enzyme.

### CROSS REFERENCE TO RELATED APPLICATIONS

This application claims the benefit of European Patent Application No. EP12156914.9, filed on Feb. 24, 2012 and European Patent Application No. EP12167393.3, filed on May 9, 2012, the disclosures of which are incorporated by reference herein in their entirety.

### FIELD OF THE INVENTION

Embodiments of the present invention relate to methods for the production of alkanes and alkenes useful in the production of biofuels and/or biochemicals, and expression vectors and host cells useful in such methods.

### BACKGROUND TO THE INVENTION

This section is intended to introduce various aspects of the art, which may be associated with exemplary embodiments of the present invention. This discussion is believed to assist in providing a framework to facilitate a better understanding of particular aspects of the present invention. Accordingly, it should be understood that this section should be read in this light, and not necessarily as admissions of any prior art.

With the diminishing supply of crude mineral oil, use of renewable energy sources is becoming increasingly important for the production of liquid fuels and/or chemicals. These fuels and/or chemicals from renewable energy sources are often referred to as biofuels. Biofuels and/or biochemicals derived from non-edible renewable energy sources are preferred as these do not compete with food production.

Hydrocarbons, such as alkanes and/or alkenes, are important constituents in the production of fuels and/or chemicals. It would therefore be desirable to produce hydrocarbons, such as alkanes and/or alkenes (sometimes also referred to as bio-alkanes and/or bio-alkenes) from non-edible renewable energy sources.

### SUMMARY

In one embodiment, there is provided a method for preparing a hydrocarbon comprising contacting a fatty acid substrate with at least one fatty acid reductase and at least one fatty aldehyde synthetase and at least one fatty acyl transferase, wherein the fatty acid substrate is a fatty acid, a fatty acyl-ACP, or a fatty acyl-CoA or a mixture of any of these, to obtain a fatty aldehyde; and contacting the fatty aldehyde with at least one aldehyde decarbonylase enzyme.

In a preferred embodiment, the method allows for the preparation of a hydrocarbon.

In one embodiment, the fatty acid reductase, the fatty aldehyde synthetase and the fatty acyl transferase can be combined in one enzyme complex, also referred to as a fatty acid reductase complex (suitably comprising at least one fatty acid reductase enzyme and at least one fatty aldehyde synthetase enzyme and at least one fatty acyl transferase enzyme). In another embodiment, the fatty acid substrate may be a fatty acid, a fatty acyl-ACP (fatty acyl-acyl carrier protein) or fatty acyl-CoA or a mixture of any of these.

In certain embodiments, the fatty acid reductase complex comprises a fatty acid reductase enzyme polypeptide having Enzyme Commission (EC) no. 1.2.1.50. In one embodiment, the fatty acid reductase enzyme has an amino acid sequence at least 50% identical to SEQ ID NO:1 (Photorhabdus luminescens protein LuxC). Additionally or independently, the fatty acid reductase complex may comprise a fatty aldehyde synthetase enzyme polypeptide having EC no. 6.2.1.19. In one embodiment, the fatty aldehydes synthetase enzyme has an amino acid sequence at least 50% identical to SEQ ID NO:2 (P. luminescens protein LuxE). Additionally or independently, the fatty acid reductase complex may comprise a fatty acyl transferase enzyme polypeptide in class EC no. 2.3.1. In one embodiment, the fatty acyl transferase enzyme has an amino acid sequence at least 50% identical to SEQ ID NO:3 (P. luminescens protein LuxD). Additionally or independently, the aldehyde decarbonylase may be in class EC 4.1.99.5. In one embodiment, the aldehydes decarbonylase has an amino acid sequence at least 50% identical to SEQ ID NO:4 (Nostoc punctiforme aldehyde decarbonylase protein). In an exemplary embodiment, all of the enzymes having the sequences SEQ ID NOs:1-4 are utilised in the method of the invention.

This summary is not intended to be a complete description of the various embodiments of the present invention. Further and alternative embodiments, and the features, aspects, and advantages of the present invention will become more apparent from the detailed descriptions, the drawings, and the claims set forth below. Further, it should be understood that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

Embodiments of the invention will now be shown, by way of example only, with reference to FIGS. 1-7 in which:

FIG. 1 is a schematic detailing the genetic elements (solid lines) introduced into E. coli cells to produce bespoke alkanes, their relationship with the endogenous genes (dashed lines) and the de novo metabolic pathway (the boxes represent genes whilst circles represent metabolic intermediates. Key to metabolites: ILV, isoleucine, leucine and valine; MDHLA, methyl-butan/propanoyl-dihydrolipoamide-E. Key to genes: ilvE, endogenous branched chain amino acid aminotransferase; E1α and E1β, branched chain alpha keto acid decarboxylase/dehydrogenase E1 α and β subunits from B. subtilis; E2, dihydrolipoyl transacylase from B. subtilis; E3, dihydrolipoamide dehydrogenase from B. subtilis (recycles lipoamide-E for use by E1 subunits); KASIII, keto-acyl synthase III (FabH2) from B. subtilis; accA to accD, endogenous acetyl-CoA carboxylase genes; fabH, endogenous beta-Ketoacyl-ACP synthase tesA, endogenous long chain thioesterase; thioesterase, Myristoyl-acyl carrier protein thioesterase from C. camphora; luxD, acyl transferase, from P. luminescens; luxC and luxE, fatty acid reductase and acyl-protein synthetase from P. luminescens; AD, aldehyde decarbonylase from N. punctiforme);

FIG. 2 shows conversion of exogenous fatty acid to alkane via the cyanobacterial alkane biosynthetic pathway. (a) GC trace of hydrocarbons extracted from E. coli BL21* (DE3) cells harbouring pACYCDuet-1 carrying the genes for NpAR in MCS1 and NpAD in MCS2; (b) GasChromatography (GC) trace of hydrocarbons extracted from E. coli BL21* (DE3) cells harbouring the cyanobacterial alkane biosynthetic plasmid described above in addition to the slr1609 from Synechocystis sp. PCC 6803 gene (peak identification: 1, methyl-pentadecane; 2, heptadecene; 3, heptadecane; 4, pentadecane; 5, unidentified);

FIG. 3 shows production of alkanes and alkenes via the novel FAR NpAD pathway. (a) composition of hydrocarbons. n=6 biological reps. Error bars represent SE mean; (b) typical GC chromatogram of alkanes extracted from E. coli cells grown in MYE media without further supplementation (top trace) or from MYE media supplemented with 13-methyl tetradecanoic acid at 100 μg/mL (bottom trace) (peak identification: 1, tridecane; 2, pentadecene; 3, pentadecane; 4, hexadecene; 5, heptadecene; 6, heptadecane; 7, methyl-tridecane);

FIG. 4 shows that expression of the camphor FatB1 thioesterase gene in E. coli increases the pool size of tetradecanoic acid. (a) GC analysis of fatty acid extracts from CEDDEC expressing cells; (b) GC analysis of fatty acid extracts from E. coli cells that expressing FatB1 (Peak identification: 1, Tetradecanoic acid; 2, Hexadecanoic acid; 3, Tetradecenoic acid; 4, Hexadecenoic acid);

FIG. 5 shows production of tridecane in E. coli cells. (a) GC trace of extracted hydrocarbons (peak identification: 1, Tridecene; 2, Tridecane; 3, Trans-5-dodecanal or tetradecanal; 4, Tridecanone; 5, Dodecanoic acid; 6, Hexadecanol); (b) MS spectral data for peak 2, tridecane;

FIG. 6 shows production of branched fatty acids in E. coli. (a) GC trace of FA extracted from control cells without BCKD/KASIII(FabH2) expression; (b) GC trace of FA extracted from cells expressing BCKD/KASIII(FabH2) (peak identification: 1, Tetradecanoic acid; 2, Hexadecanoic acid; 3, methyl-Tetradecanoic acid; 4, methyl-Hexadecanoic acid; 5, methyl-Hexadecanoic acid); and

FIG. 7 shows production of branched pentadecane in E. coli cells. (a) Typical GC trace (peak identification: 1, Pentadecane; 2, methyl-Pentadecane; 3, Hexadecene; 4, Heptadecene); (b) Mass spectral data for peak 2, methyl-pentadecane.

### DETAILED DESCRIPTION OF THE PREFERRED EMBODIMENTS

Unless otherwise defined herein, scientific and technical terms used herein will have the meanings that are commonly understood by those of ordinary skill in the art.

Generally, nomenclatures used in connection with techniques of biochemistry, enzymology, molecular and cellular biology, microbiology, genetics and protein and nucleic acid chemistry and hybridization, described herein, are those well known and commonly used in the art.

Conventional methods and techniques mentioned herein are explained in more detail, for example, in Molecular Cloning, a laboratory manual [second edition] Sambrook et al. Cold Spring Harbor Laboratory, 1989, for example in Sections 1.21 “Extraction And Purification Of Plasmid DNA”, 1.53 “Strategies For Cloning In Plasmid Vectors”, 1.85 “Identification Of Bacterial Colonies That Contain Recombinant Plasmids”, 6 “Gel Electrophoresis Of DNA”, 14 “In vitro Amplification Of DNA By The Polymerase Chain Reaction”, and 17 “Expression Of Cloned Genes In Escherichia coli” thereof.

The identity of amino acid and nucleotide sequences referred to in this specification is as set out in Table 4 at the end of the description. The terms “polynucleotide”, “polynucleotide sequence” and “nucleic acid sequence” are used interchangeably herein. The terms “polypeptide”, “polypeptide sequence” and “amino acid sequence” are, likewise, used interchangeably herein. Other exemplary sequences encompassed by certain embodiments of the invention are provided in the Sequence Listing.

Enzyme Commission (EC) numbers (also called “classes” herein), referred to throughout this specification, are according to the Nomenclature Committee of the International Union of Biochemistry and Molecular Biology (NC-IUBMB) in its resource “Enzyme Nomenclature” (1992, including Supplements 6-17) available, for example, as “Enzyme nomenclature 1992: recommendations of the Nomenclature Committee of the International Union of Biochemistry and Molecular Biology on the nomenclature and classification of enzymes”, Webb, E. C. (1992), San Diego: Published for the International Union of Biochemistry and Molecular Biology by Academic Press (ISBN 0-12-227164-5). This is a numerical classification scheme based on the chemical reactions catalysed by each enzyme class.

The fatty aldehyde may herein also be referred to as fatty aldehyde hydrocarbon precursor. The term “fatty aldehyde hydrocarbon precursor” indicates a fatty aldehyde compound which can be used as a hydrocarbon precursor. In other words, in the method according to one of the embodiments of the invention a fatty aldehyde is prepared, which fatty aldehyde may subsequently be converted into a hydrocarbon.

The term “fatty acid reductase complex” may comprise an enzyme complex capable of catalysing the conversion of free fatty acid, fatty acyl-ACP or fatty acyl-CoA to fatty aldehyde. Preferably, the complex comprises a fatty acid reductase enzyme and a fatty aldehyde synthetase enzyme and a fatty acyl transferase enzyme. The term “fatty aldehyde synthetase” indicates an enzyme in class EC 6.2.1.19 capable of catalysing the formation of an acyl-protein thioester from a fatty acid and a protein. The term “fatty acid reductase” indicates an enzyme in class EC 1.2.1.50, the enzyme being capable of catalysing the formation of a long-chain aldehyde from a fatty acyl-AMP (fatty acyl-adenosine monophosphate) or a fatty acyl-CoA. Fatty acyl-AMP is the intermediate formed by the fatty aldehyde synthetase in this coupled reaction. An example of a fatty acid reductase is the polypeptide having amino acid sequence SEQ ID NO:1; an example of a fatty aldehyde synthetase is the polypeptide having amino acid sequence SEQ ID NO:2. Other suitable fatty acid reductase polypeptides have amino acid sequence at least 50% identical to SEQ ID NO:1, e.g., SEQ ID NO:28 or 29; other suitable fatty aldehyde synthetase polypeptides have an amino acid sequence at least 50% identical to SEQ ID NO:2, e.g., SEQ ID NO:32 or 33.

The term “fatty acyl transferase” indicates an enzyme in class EC 2.3.1, capable of catalysing the transfer of the acyl moiety of fatty acyl-ACP, acyl-CoA and other activated acyl donors, to the hydroxyl group of a serine on the transferase, followed by the conversion of the ester to a fatty acid through hydrolysis. An example of a fatty acyl transferase is the polypeptide having amino acid sequence SEQ ID NO:3. Other suitable fatty acyl transferase polypeptides have an amino acid sequence at least 50% identical to SEQ ID NO:3, e.g. SEQ ID NO:30 or 31.

The term “aldehyde decarbonylase” indicates an enzyme in class EC 4.1.99.5, capable of catalysing the conversion of fatty aldehyde to a hydrocarbon, for example an alkane, alkene or mixture thereof. An example of an aldehyde decarbonylase is the polypeptide having amino acid sequence SEQ ID NO:4 or an amino acid sequence at least 50% identical to SEQ ID NO:4.

The terms “fatty aldehyde synthetase”, “fatty aldehyde synthetase enzyme”, “fatty aldehyde synthetase enzyme polypeptide” and “fatty aldehyde synthetase polypeptide” are used interchangeably herein.

The terms “fatty acid reductase”, “fatty acid reductase enzyme”, “fatty acid reductase enzyme polypeptide” and “fatty acid reductase polypeptide” are used interchangeably herein.

The terms “fatty acyl transferase”, “fatty acyl transferase enzyme”, “fatty acyl transferase enzyme polypeptide” and “fatty acyl transferase polypeptide” are used interchangeably herein.

The terms “aldehyde carbonylase”, “aldehyde carbonylase enzyme”, “aldehyde carbonylase enzyme polypeptide” and “aldehyde carbonylase polypeptide” are used interchangeably herein.

The term “Folch method” refers to the method for extraction described by Folch et al. in their article titled “Preparation of blood lipid extracts free from non-lipid extractives”, published in Proc. Soc. Exp. Biol. Med. 41 (2), 514-515 (1939) (herein incorporated by reference).

In one embodiment, the enzymes described above are active in the temperature range 0-60° C., for example in the range 10-50° C. In an embodiment, at least the fatty acid reductase, fatty aldehyde synthetase and fatty acyl transferase enzymes have significant (i.e., detectable) activity at about 45° C.

In an embodiment of the method according to the first aspect of the invention, at least some of the fatty acid is obtainable by contacting a fatty acyl-ACP with at least one acyl-ACP thioesterase. The term acyl-ACP thioesterase is an enzyme in the class EC 3.1.2.14, capable of catalysing the release of free fatty acid from fatty acyl-ACP. The acyl-ACP thioesterase may be, for example, a polypeptide having at least 50% sequence identity to SEQ ID NO:5 (thioesterase protein from Cinnamomum camphora).

In an embodiment of the method, at least some of the fatty acyl-ACP mentioned in any preceding embodiment is obtainable by contacting a keto acyl CoA and a malonyl-ACP with at least one 3-ketoacyl-ACP synthase III (KASIII). This is an enzyme in class EC 2.3.1.180, capable of catalysing the reaction of a keto acyl CoA and a malonyl-ACP to form fatty acyl-ACP. The 3-ketoacyl-ACP synthase III may be a polypeptide having at least 50% sequence identity to SEQ ID NO:6 (Bacillus subtilis enzyme KASIII).

In this embodiment, at least some of the keto acyl-CoA may be obtainable by contacting a keto acid with a branched-chain ketodehydrogenase complex. This is an enzyme or complex of enzymes capable of catalysing the conversion of a keto acid to a keto acyl-CoA. For example, the branched-chain ketodehydrogenase complex may comprise a polypeptide in class EC 1.2.4.4 (for example having at least 50% sequence identity to SEQ ID NO:7; B. subtilis BCKD subunit E1α) and a further polypeptide in class EC 1.2.4.4 (for example having at least 50% sequence identity to SEQ ID NO:8; B. subtilis BCKD subunit E1β and a polypeptide in class EC 2.3.1.168 (for example having at least 50% sequence identity to SEQ ID NO:9; B. subtilis BCKD subunit E2) and a polypeptide in class EC 1.8.1.4 (for example having at least 50% sequence identity to SEQ ID NO:10; B. subtilis BCKD subunit E3). In an embodiment, the branched-chain ketodehydrogenase complex is a single polypeptide comprising all of the amino acid sequences SEQ ID NOs:7-10.

A hydrocarbon is an organic compound containing hydrogen and carbon and, more preferably, an organic compound consisting entirely of hydrogen and carbon. Examples of hydrocarbons containing hydrogen and carbon in embodiments of the invention include alkanes, alkenes and/or mixtures thereof. Preferably the alkanes and/or alkenes are linear or branched alkanes and/or alkenes. The hydrocarbon may be a single alkane or a single alkene, or may be a mixture of at least two alkanes and/or a mixture of at least two alkenes and/or a mixture of at least one alkane and at least one alkene. As is well known to the skilled person, an alkane is a hydrocarbon in which the atoms are linked together exclusively by single bonds (i.e., they are saturated compounds). Examples of suitable alkanes produced using an embodiment of the invention have between 4 and 30 carbon atoms, more preferably between 8 and 18 carbon atoms, in linear or branched configuration, for example, heptadecane, pentadecane and methyl-heptadecane. As is again well known to the skilled person, an alkene is an unsaturated hydrocarbon comprising at least one carbon-to-carbon double bond. Examples of suitable alkenes produced using an embodiment of the invention have between 4 and 30 carbon atoms, more preferably between 8 and 18 carbon atoms, in linear or branched formation and comprise one or more double bonds. Particular examples of alkanes and/or alkenes produced using an embodiment of the invention included straight- or branched-chain alkanes and/or alkenes having up to 2, 3, 4, 5, 6, 7, 8, 9, 10, 11, 12, 13, 14, 15, 16, 17, 18, 19 or up to 20 carbon atoms.

The method may subsequently comprise isolating the hydrocarbon. The term “isolating the hydrocarbon” indicates that the hydrocarbon (i.e., alkane, alkene or mixture thereof) is separated from other non-hydrocarbon components, such as any cell lysate components which may be present at the end of the method of the first aspect of the invention. This may indicate that, for example, at least about 50% by weight of a sample after isolating the hydrocarbon is composed of the hydrocarbon(s) at a percentage of, for example, at least about 60%, 70%, 75%, 80%, 85%, 90%, 95%, 96%, 97%, 98%, 99% or 100%. The hydrocarbons produced during the working of the invention can be separated (i.e., isolated) by any known technique. One exemplary process is a two-phase (bi-phasic) separation process, involving conducting the method for a period and/or under conditions sufficient to allow the hydrocarbon(s) to collect in an organic phase and separating the organic phase from an aqueous phase. This may be especially relevant when, for example, the method is conducted within a host cell such as a micro-organism, as described below. Bi-phasic separation uses the relative immiscibility of hydrocarbons to facilitate separation. “Immiscible” refers to the relative inability of a compound to dissolve in water and is defined by the compound's partition coefficient, as will be well understood by the skilled person.

A fatty acid (FA) is a carboxylic acid with a long unbranched or branched aliphatic tail. The fatty acid can comprise saturated fatty acids and/or unsaturated fatty acids containing one, two, three or more double bonds. The one or more fatty acid(s), fatty acyl-ACP or fatty acyl-CoA may, for example, comprise 4 or more carbon atoms, for example, 8 or more carbon atoms, 10 or more carbon atoms, 12 or more carbon atoms, or 14 or more carbon atoms. The fatty acid may also comprise, for example, 30 or fewer carbon atoms, for example, 26 or fewer carbon atoms, 25 or fewer carbon atoms, 23 or fewer carbon atoms, or 20 or fewer carbon atoms. Preferably the one or more fatty acid(s), fatty acyl-ACP and/or fatty acyl-CoA may comprise in the range from 8 or more carbon atoms to 30 or fewer carbon atoms, preferably to 20 or fewer carbon atoms, most preferably to 18 or fewer carbon atoms. Fatty acids may, for example, be derived from triacylglycerols or phospholipids, or may be made de novo by a cell, and/or by mechanisms described elsewhere herein.

In an embodiment of the invention, the fatty acid reductase and the fatty aldehyde synthetase and the fatty acyl transferase and the aldehyde decarbonylase enzymes are expressed by a recombinant host cell, such as a recombinant micro-organism. Therefore, the steps of the first aspect of the invention may take place within a host cell, i.e., the method may be at least partially an in vivo method. The host cell may be recombinant and may, for example, be a genetically modified microorganism. Therefore, a micro-organism may be genetically modified, i.e., artificially altered from its natural state, to express at least one of the fatty acid reductase, fatty aldehyde synthetase and fatty acyl transferase enzymes and, preferably, all of these. It may also express the aldehyde decarbonylase enzyme. Other enzymes described herein (i.e., an acyl-ACP thioesterase and/or a 3-ketoacyl-ACP synthase III and/or a branched-chain ketodehydrogenase complex) may also be expressed by a micro-organism. Preferably, the enzymes are exogenous, i.e., not present in the cell prior to modification, having been introduced using microbiological methods such as are described herein. Furthermore, in the method of the invention, the enzymes may each be expressed by a recombinant host cell, either within the same host cell or in separate host cells. The hydrocarbon may be secreted from the host cell in which it is formed.

The host cell may be genetically modified by any manner known to be suitable for this purpose by the person skilled in the art. This includes the introduction of the genes of interest, such as one or more genes encoding the fatty acid reductase and/or the fatty aldehyde synthetase and/or the fatty acyl transferase and/or the aldehyde decarbonylase and/or the acyl-ACP thioesterase and/or the 3-ketoacyl-ACP synthase III and/or the branched-chain ketodehydrogenase complex enzymes, on a plasmid or cosmid or other expression vector which may be capable of reproducing within the host cell. Alternatively, the plasmid or cosmid DNA or part of the plasmid or cosmid DNA or a linear DNA sequence may integrate into the host genome, for example by homologous recombination. To carry out genetic modification, DNA can be introduced or transformed into cells by natural uptake or mediated by well-known processes such as electroporation. Genetic modification can involve expression of a gene under control of an introduced promoter. The introduced DNA may encode a protein which could act as an enzyme or could regulate the expression of further genes.

Such a host cell may comprise a nucleic acid sequence encoding a fatty acid reductase and/or a fatty aldehyde synthetase and/or a fatty acyl transferase and/or an aldehyde decarbonylase and/or an acyl-ACP thioesterase and/or a 3-ketoacyl-ACP synthase III and/or a branched-chain ketodehydrogenase complex. For example, the cell may comprise at least one nucleic acid sequence comprising at least one of the polynucleotide sequences SEQ ID NOs:11-24 or a complement thereof, or a fragment of such a polynucleotide encoding a functional variant (which may be a fragment providing a functional variant) of any of the enzymes fatty acid reductase and/or fatty aldehyde synthetase and/or fatty acyl transferase and/or aldehyde decarbonylase and/or acyl-ACP thioesterase and/or 3-ketoacyl-ACP synthase III and/or branched-chain ketodehydrogenase complex, for example enzymes as described herein. The nucleic acid sequences encoding the enzymes may be exogenous, i.e., not naturally occurring in the host cell.

Therefore, a second aspect of the invention provides a recombinant host cell, such as a micro-organism, comprising at least one polypeptide which is a fatty acid reductase in class EC 1.2.1.50, for example, having an amino acid sequence at least 50% identical to SEQ ID NO:1 (e.g., SEQ ID NO:1, 28 or 29), and comprising at least one polypeptide which is a fatty aldehyde synthetase in class EC 6.2.1.19, for example, having an amino acid sequence at least 50% identical to SEQ ID NO:2 (e.g., SEQ ID NO:2, 32 or 33), and comprising at least one polypeptide which is a fatty acyl transferase in class EC 2.3.1, for example, having an amino acid sequence at least 50% identical to SEQ ID NO:3 (e.g., SEQ ID NO:3, 30 or 31). The cell may also comprise at least one polypeptide which is an aldehyde decarbonylase in class EC 4.1.99.5, for example, having an amino acid sequence at least 50% identical to SEQ ID NO:4, or a functional variant or fragment of any of these sequences. The recombinant host cell may comprise a polypeptide comprising all of SEQ ID NOs:1-4 and/or amino acid sequences at least 50% identical to all of SEQ ID NOs:1-3 (e.g., amino acid sequences selected from SEQ ID NOs:28-33, as outlined above) and at least 50% identical to SEQ ID NO:4. The recombinant host cell may comprise the polynucleotide sequences SEQ ID NOs:11-14 and/or the sequences SEQ ID NOs:13 & 15 and/or the sequences SEQ ID NOs:13-16 and/or any combination of these specific combinations.

The recombinant host cell may further comprise: at least one acyl-ACP thioesterase in class EC 3.1.2.14 (e.g., having an amino acid sequence which is at least 50% identical to any of SEQ ID NOs:5 or a functional variant or fragment thereof); and/or at least one 3-ketoacyl-ACP synthase III in class EC 2.3.1.180 (e.g., having an amino acid sequence which is at least 50% identical to any of SEQ ID NOs:6 or a functional variant or fragment thereof); and/or at least one branched-chain ketodehydrogenase complex comprising enzymes in classes EC 1.2.4.4, 2.3.1.168 and 1.8.1.4 (e.g., comprising one or more amino acid sequence(s) each being at least 50% identical to any of SEQ ID NOs:7-10 or a functional variant or fragment thereof); and/or at least one polynucleotide encoding at least one of these enzymes and/or functional fragments or variants of these. The cell may also be modified to produce increased levels of fatty acid which may be used by the fatty acid reductase and fatty aldehyde synthetase and fatty acyl transferase as a substrate to form a fatty aldehyde which may then be converted to a hydrocarbon by the decarbonylase. The recombinant host cell may also comprise one or more transport proteins for transporting hydrocarbon(s) out of the cell.

A suitable polynucleotide may be introduced into the cell by homologous recombination and/or may form part of an expression vector comprising at least one of the polynucleotide sequences SEQ ID NOs:11-25 or a complement thereof. Such an expression vector forms a third aspect of the invention. Suitable vectors for construction of such an expression vector are well known in the art (examples are mentioned above) and may be arranged to comprise the polynucleotide operably linked to one or more expression control sequences, so as to be useful to express the required enzymes in a host cell, for example a micro-organism as described above.

In some embodiments, the recombinant or genetically modified host cell, as mentioned throughout this specification, may be any micro-organism or part of a micro-organism selected from the group consisting of fungi (such as members of the genus Saccharomyces), protists, algae, bacteria (including cyanobacteria) and archaea. The bacterium may comprise a gram-positive bacterium or a gram-negative bacterium and/or may be selected from the genera Escherichia, Bacillus, Lactobacillus, Rhodococcus, Pseudomonas or Streptomyces. The cyanobacterium may be selected from the group of Synechococcus elongatus, Synechocystis, Prochlorococcus marinus, Anabaena variabilis, Nostoc punctiforme, Gloeobacter violaceus, Cyanothece sp. and Synechococcus sp. The selection of a suitable micro-organism (or other expression system) is within the routine capabilities of the skilled person. Particularly suitable micro-organisms include Escherichia coli and Saccharomyces cerevisiae, for example.

In a related embodiment of the invention, a fatty acid reductase and/or a fatty aldehyde synthetase and/or a fatty acyl transferase and/or an aldehyde decarbonylase and/or an acyl-ACP thioesterase and/or a 3-ketoacyl-ACP synthase III and/or a branched-chain ketodehydrogenase complex or functional variant or functional fragment of any of these may be expressed in a non-micro-organism cell such as a cultured mammalian cell or a plant cell or an insect cell. Mammalian cells may include CHO cells, COS cells, VERO cells, BHK cells, HeLa cells, Cvl cells, MDCK cells, 293 cells, 3T3 cells, and/or PC12 cells.

The recombinant host cell or micro-organism may be used to express the enzymes mentioned above and a cell-free extract then obtained by standard methods, for use in the method according to the first aspect of the invention.

Embodiments of the present invention also encompass variants of the polypeptides as defined herein. As used herein, a “variant” means a polypeptide in which the amino acid sequence differs from the base sequence from which it is derived in that one or more amino acids within the sequence are substituted for other amino acids. For example, a variant of SEQ ID NO:1 may have an amino acid sequence at least about 50% identical to SEQ ID NO:1, for example, at least about 55%, 60%, 65%, 70%, 75%, 80%, 85%, 90%, 91%, 92%, 93%, 94%, 95%, 96%, 97%, 98%, 99% or about 100% identical. The variants and/or fragments are functional variants/fragments in that the variant sequence has similar or identical functional enzyme activity characteristics to the enzyme having the non-variant amino acid sequence specified herein (and this is the meaning of the term “functional variant” as used throughout this specification).

For example, a functional variant of SEQ ID NO:1 has similar or identical fatty acid reductase characteristics as SEQ ID NO:1, being classified in enzyme class EC 1.2.1.50 by the Enzyme Nomenclature of NC-IUBMB as mentioned above. An example may be that the rate of conversion by a functional variant of SEQ ID NO:1, in the presence of non-variant SEQ ID NO:2, of a free fatty acid to fatty aldehyde may be the same or similar, for example at least about 60%, 70%, 80%, 90%, 95%, 96%, 97%, 98%, 99% or at least about 100% the rate achieved when using the enzyme having amino acid sequence SEQ ID NO:1, in the presence of non-variant SEQ ID NO:2. The rate may be improved when using the variant polypeptide, so that a rate of more than 100% the non-variant rate is achieved. Equivalent analysis of percentage sequence identity and comparative functional variant activity may, likewise, be made for other enzymes mentioned herein.

For example, a variant of the fatty acyl transferase SEQ ID NO:3 may have an amino acid sequence at least about 50% identical to SEQ ID NO:3, being a functional variant in that it is classified in EC 2.3.1; the rate of transfer of the acyl moiety of fatty acyl-ACP, acyl-CoA and other activated acyl donors, to the hydroxyl group of a serine on the transferase, followed by the conversion of the ester to a fatty acid through hydrolysis, may be the same or similar, for example at least about 60%, 70%, 80%, 90% or 95% the rate achieved when using SEQ ID NO:3.

SEQ ID NOs:28 and 29 may be examples of functional variants of SEQ ID NO:1, as defined herein. SEQ ID NOs:32 and 33 may be examples of functional variants of SEQ ID NO:2, as defined herein. SEQ ID NOs:30 and 31 may be examples of functional variants of SEQ ID NO:3, as defined herein.

The NC-IUBMB classification of the enzymes mentioned herein is, in summary, set out in Table 1 below.

TABLE 1

| | SEQ ID | EC |
|---|---|---|
| Description of sequence | NO | number |
| Photorhabdus luminescens LuxC amino acid | 1 | 1.2.1.50 |
| sequence | | |
| P. luminescens LuxE amino acid sequence | 2 | 6.2.1.19 |
| P. luminescens LuxD amino acid sequence | 3 | 2.3.1.— |
| Nostoc punctiforme aldehyde decarbonylase | 4 | 4.1.99.5 |
| amino acid sequence | | |
| Cinnamomum camphora thioesterase amino | 5 | 3.1.2.14 |
| acid sequence | | |
| Bacillus subtilis KasIII (3-ketoacyl-ACP | 6 | 2.3.1.180 |
| synthase III) amino acid sequence | | |
| B. subtilis BCKD subunit E1α amino acid sequence | 7 | 1.2.4.4 |
| B. subtilis BCKD subunit E1β amino acid sequence | 8 | 1.2.4.4 |
| B. subtilis BCKD subunit E2 amino acid sequence | 9 | 2.3.1.168 |
| B. subtilis BCKD subunit E3 amino acid sequence | 10 | 1.8.1.4 |

A functional variant or fragment of any of the above SEQ ID NO amino acid sequences, therefore, is any amino acid sequence which remains within the same enzyme category (i.e., has the same EC number) as the non-variant sequences as set out in Table 1. Methods of determining whether an enzyme falls within a particular category are well known to the skilled person, who can determine the enzyme category without use of inventive skill Suitable methods may, for example, be obtained from the International Union of Biochemistry and Molecular Biology.

Amino acid substitutions may be regarded as “conservative” where an amino acid is replaced with a different amino acid with broadly similar properties. Non-conservative substitutions are where amino acids are replaced with amino acids of a different type.

By “conservative substitution” is meant the substitution of an amino acid by another amino acid of the same class, in which the classes are defined as follows:

### Class Amino Acid Examples

### Nonpolar: A, V, L, I, P, M, F, W

Uncharged polar: G, S, T, C, Y, N, Q

### Acidic: D, E

### Basic: K, R, H.

As is well known to those skilled in the art, altering the primary structure of a polypeptide by a conservative substitution may not significantly alter the activity of that polypeptide because the side-chain of the amino acid which is inserted into the sequence may be able to form similar bonds and contacts as the side chain of the amino acid which has been substituted out. This is so even when the substitution is in a region which is critical in determining the polypeptide's conformation.

In embodiments of the present invention, non-conservative substitutions are possible provided that these do not interrupt the enzyme activities of the polypeptides, as defined elsewhere herein. The substituted versions of the enzymes must retain characteristics such that they remain in the same enzyme class as the non-substituted enzyme, as determined using the NC-IUBMB nomenclature discussed above.

Broadly speaking, fewer non-conservative substitutions than conservative substitutions will be possible without altering the biological activity of the polypeptides. Determination of the effect of any substitution (and, indeed, of any amino acid deletion or insertion) is wholly within the routine capabilities of the skilled person, who can readily determine whether a variant polypeptide retains the enzyme activity according to aspects of the invention. For example, when determining whether a variant of the polypeptide falls within the scope of the invention (i.e., is a “functional variant or fragment” as defined above), the skilled person will determine whether the variant or fragment retains the substrate converting enzyme activity as defined with reference to the NC-IUBMB nomenclature mentioned elsewhere herein. All such variants are within the scope of the invention.

Using the standard genetic code, further nucleic acid sequences encoding the polypeptides may readily be conceived and manufactured by the skilled person, in addition to those disclosed herein. The nucleic acid sequence may be DNA or RNA, and where it is a DNA molecule, it may for example comprise a cDNA or genomic DNA. The nucleic acid may be contained within an expression vector, as described elsewhere herein.

Embodiments of the invention, therefore, encompass variant nucleic acid sequences encoding the polypeptides contemplated by embodiments of the invention. The term “variant” in relation to a nucleic acid sequence means any substitution of, variation of, modification of, replacement of, deletion of, or addition of one or more nucleotide(s) from or to a polynucleotide sequence, providing the resultant polypeptide sequence encoded by the polynucleotide exhibits at least the same or similar enzymatic properties as the polypeptide encoded by the basic sequence. The term includes allelic variants and also includes a polynucleotide (a “probe sequence”) which substantially hybridises to the polynucleotide sequence of embodiments of the present invention. Such hybridisation may occur at or between low and high stringency conditions. In general terms, low stringency conditions can be defined as hybridisation in which the washing step takes place in a 0.330-0.825 M NaCl buffer solution at a temperature of about 40-48° C. below the calculated or actual melting temperature (Tₘ) of the probe sequence (for example, about ambient laboratory temperature to about 55° C.), while high stringency conditions involve a wash in a 0.0165-0.0330 M NaCl buffer solution at a temperature of about 5-10° C. below the calculated or actual Tₘ of the probe sequence (for example, about 65° C.). The buffer solution may, for example, be SSC buffer (0.15M NaCl and 0.015M tri-sodium citrate), with the low stringency wash taking place in 3×SSC buffer and the high stringency wash taking place in 0.1×SSC buffer. Steps involved in hybridisation of nucleic acid sequences have been described for example in Molecular Cloning, a laboratory manual [second edition] Sambrook et al. Cold Spring Harbor Laboratory, 1989, for example in Section 11 “Synthetic Oligonucleotide Probes” thereof (herein incorporated by reference)

Preferably, nucleic acid sequence variants have about 55% or more of the nucleotides in common with the nucleic acid sequence of embodiments of the present invention, more preferably 60%, 65%, 70%, 80%, 85%, or even 90%, 95%, 98% or 99% or greater sequence identity.

Variant nucleic acids of the invention may be codon-optimised for expression in a particular host cell.

Sequence identity between amino acid sequences can be determined by comparing an alignment of the sequences using the Needleman-Wunsch Global Sequence Alignment Tool available from the National Center for Biotechnology Information (NCBI), Bethesda, Md., USA, for example via http://blast.ncbi.nlm.nih.gov/Blast.cgi, using default parameter settings (for protein alignment, Gap costs Existence:11 Extension:1). Sequence comparisons and percentage identities mentioned in this specification have been determined using this software. When comparing the level of sequence identity to, for example, SEQ ID NO:1, this, preferably should be done relative to the whole length of SEQ ID NO:1 (i.e., a global alignment method is used), to avoid short regions of high identity overlap resulting in a high overall assessment of identity. For example, a short polypeptide fragment having, for example, five amino acids might have a 100% identical sequence to a five amino acid region within the whole of SEQ ID NO:1, but this does not provide a 100% amino acid identity unless the fragment forms part of a longer sequence which also has identical amino acids at other positions equivalent to positions in SEQ ID NO:1. When an equivalent position in the compared sequences is occupied by the same amino acid, then the molecules are identical at that position. Scoring an alignment as a percentage of identity is a function of the number of identical amino acids at positions shared by the compared sequences. When comparing sequences, optimal alignments may require gaps to be introduced into one or more of the sequences, to take into consideration possible insertions and deletions in the sequences. Sequence comparison methods may employ gap penalties so that, for the same number of identical molecules in sequences being compared, a sequence alignment with as few gaps as possible, reflecting higher relatedness between the two compared sequences, will achieve a higher score than one with many gaps. Calculation of maximum percent identity involves the production of an optimal alignment, taking into consideration gap penalties. As mentioned above, the percentage sequence identity may be determined using the Needleman-Wunsch Global Sequence Alignment tool, using default parameter settings. The Needleman-Wunsch algorithm was published in J. Mol. Biol. (1970) vol. 48:443-53.

Polypeptide and polynucleotide sequences for use in the methods, vectors and host cells according to embodiments of the invention are shown in the Sequence Listing.

According to a fourth aspect of the invention, there is provided a method of producing an alkane, comprising hydrogenation of an isolated alkene produced in a method according to the first aspect of the invention.

The unsaturated bonds in the isolated alkene can be hydrogenated to produce the alkane. Hydrogenation may be carried out in any manner known by the person skilled in the art to be suitable for hydrogenation of unsaturated compounds. The hydrogenation catalyst can be any type of hydrogenation catalyst known by the person skilled in the art to be suitable for this purpose. The hydrogenation catalyst may comprise one or more hydrogenation metal(s), for example, supported on a catalyst support. The one or more hydrogenation metal(s) may be chosen from Group VIII and/or Group VIB of the Periodic Table of Elements. The hydrogenation metal may be present in many forms; for example, it may be present as a mixture, alloy or organometallic compound. The one or more hydrogenation metal(s) may be chosen from the group consisting of Nickel (Ni), Molybdenum (Mo), Tungsten (W), Cobalt (Co) and mixtures thereof. The catalyst support may comprise a refractory oxide or mixtures thereof, for example, alumina, amorphous silica-alumina, titania, silica, ceria, zirconia; or it may comprise an inert component such as carbon or silicon carbide.

The temperature for hydrogenation may range from, for example, 300° C. to 450° C., for example, from 300° C. to 350° C. The pressure may range from, for example, 50 bar absolute to 100 bar absolute, for example, 60 bar absolute to 80 bar absolute.

A fifth aspect of the invention provides a method of producing a branched alkane, comprising hydroisomerization of an isolated alkene and/or alkane produced in a method according to the first aspect of the invention. Hydroisomerization may be carried out in any manner known by the person skilled in the art to be suitable for hydroisomerization of alkanes. The hydroisomerization catalyst can be any type of hydroisomerization catalyst known by the person skilled in the art to be suitable for this purpose. The one or more hydrogenation metal(s) may be chosen from Group VIII and/or Group VIB of the Periodic Table of Elements. The hydrogenation metal may be present in many forms, for example it may be present as a mixture, alloy or organometallic compound. The one or more hydrogenation metal(s) may be chosen from the group consisting of Nickel (Ni), Molybdenum (Mo), Tungsten (W), Cobalt (Co) and mixtures thereof. The catalyst support may comprise a refractory oxide, a zeolite, or mixtures thereof. Examples of catalyst supports include alumina, amorphous silica-alumina, titania, silica, ceria, zirconia; and zeolite Y, zeolite beta, ZSM-5, ZSM-12, ZSM-22, ZSM-23, ZSM-48, SAPO-11, SAPO-41, and ferrierite.

Hydroisomerization may be carried out at a temperature in the range of, for example, from 280 to 450° C. and a total pressure in the range of, for example, from 20 to 160 bar (absolute).

In one embodiment hydrogenation and hydroisomerization are carried out simultaneously.

A sixth aspect of the invention provides a method for the production of a biofuel and/or a biochemical comprising combining an alkene and/or alkane produced in a method according to the first aspect of the invention with one or more additional components to produce a biofuel and/or biochemical.

According to a seventh aspect of the invention, there is provided a method for the production of a biofuel and/or a biochemical comprising combining an alkane produced according to the fourth or fifth aspects with one or more additional components to produce a biofuel and/or biochemical.

In the sixth and seventh aspects, the alkane and/or alkene can be blended as a biofuel component and/or a biochemical component with one or more other components to produce a biofuel and/or a biochemical. By a biofuel or a biochemical, respectively, is herein understood a fuel or a chemical that is at least partly derived from a renewable energy (i.e., non-fossil fuel) source. Examples of one or more other components with which alkane and/or alkene may be blended include anti-oxidants, corrosion inhibitors, ashless detergents, dehazers, dyes, lubricity improvers and/or mineral fuel components, but also conventional petroleum-derived gasoline, diesel and/or kerosene fractions.

A further aspect of the invention provides the use of a host cell according to the second aspect of the invention as a biofuel/biochemical hydrocarbon precursor source. A “biofuel/biochemical hydrocarbon precursor” is a hydrocarbon, preferably an alkane, alkene or mixture thereof, which may be used in the preparation of a biofuel and/or a biochemical, for example in a method according to the sixth or seventh aspects of the invention. The use of a host cell as the source of such a precursor indicates that the host cell according to the second aspect of the invention produces hydrocarbons suitable for use in the biofuel/biochemical production methods, the hydrocarbons being isolatable from the recombinant host cell as described elsewhere herein.

Throughout the description and claims of this specification, the words “comprise” and “contain” and variations of the words, for example “comprising” and “comprises”, mean “including but not limited to” and do not exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Preferred features of each aspect of the invention may be as described in connection with any of the other aspects.

Other features of the present invention will become apparent from the following examples. Generally speaking, the invention extends to any novel one, or any novel combination, of the features disclosed in this specification (including the accompanying claims and drawings). Thus, features, integers, characteristics, compounds or chemical moieties described in conjunction with a particular aspect, embodiment or example of the invention are to be understood to be applicable to any other aspect, embodiment or example described herein, unless incompatible therewith.

Moreover, unless stated otherwise, any feature disclosed herein may be replaced by an alternative feature serving the same or a similar purpose.

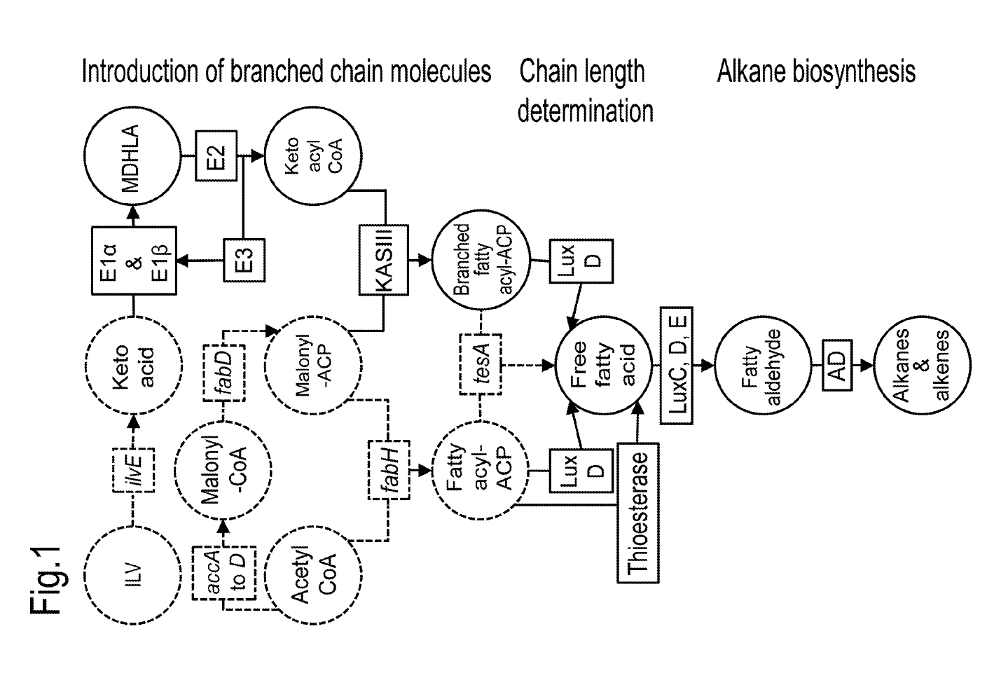

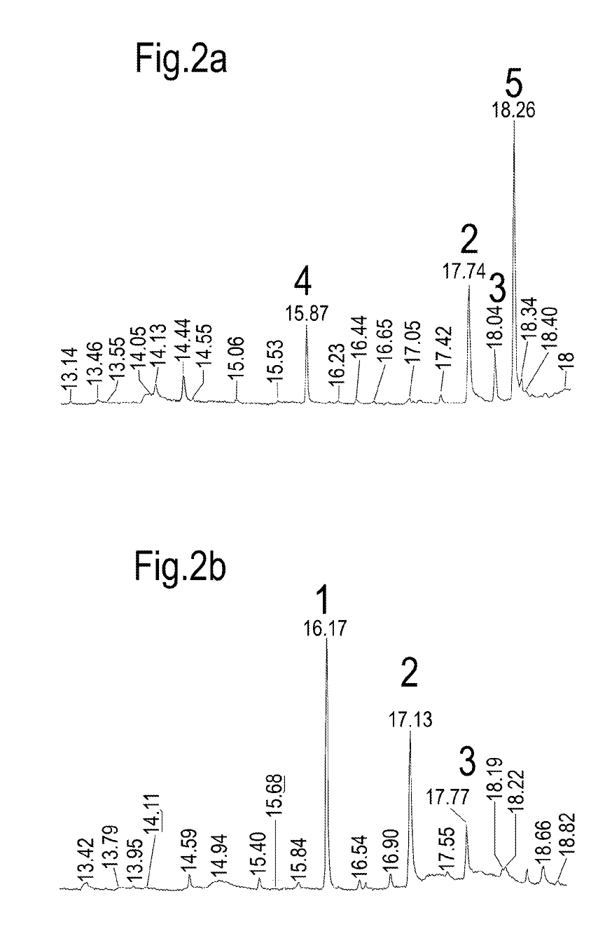

In order to achieve production of metabolically-derived, fuel-grade hydrocarbons, the inventors designed an alkane biosynthetic pathway de novo (FIG. 1). The aim was two-fold: to demonstrate the sole production of fuel-grade chain length alkanes in E. coli (i.e., less than 16 carbon chain length alkanes) and to demonstrate the production of branched-chain alkanes in E. coli. In order to achieve the first aim, the inventors sought to synthesise alkanes from a modified fatty acid (Fatty acid is herein also abbreviated to FA) substrate pool. Targeting the FA pool would enable the introduction of thioesterase activity to modify alkane chain length. One of the possible problems, however, is that introduction of thioesterase activity is not compatible with the cyanobacterial alkane biosynthetic pathway. This is because thioesterase activity releases free FAs of differing chain length from fatty acyl-ACP; free FAs are not an entry substrate for the cyanobacterial alkane pathway and need to be re-activated to the corresponding fatty acyl-ACP for use by an acyl-ACP reductase (AR) (see also the article of Schirmer et al., titled “Microbial biosynthesis of alkanes”, published in Science volume 329 (5991), pages 559-562 (2010) herein incorporated by reference). Whilst this can be accomplished through expression of the cyanobacterial gene slr1609 from Synechocystis sp. PCC 6803 as for example described in the article by Kaczmarzyk et al. titled “Fatty acid activation in Cyanobacteria mediated by acyl-acyl carrier protein synthetase enables fatty acid recycling” Plant Physiol. vol 152 (3), pages 1598-1610 (2010) herein incorporated by reference) (FIG. 2), such a modification is undesirable. This is because it is likely to create a futile cycle between slr1609 from Synechocystis sp. PCC 6803 and any introduced thioesterase activity and furthermore, activated short chain fatty-acyl substrates may simply re-enter the FA elongation cycle.

Instead, the inventors hypothesised that the fatty acid reductase (FAR) complex from the bacterial luciferase operon (see also for example the article of Meighen titled “Molecular biology of bacterial bioluminescence” published in Microbiol. Rev. 55 (1), pages 123-142 (1991) herein incorporated by reference) would supply fatty aldehyde substrate to an aldehyde decarbonylase (AD) reaction (such as that recently described from cyanobacteria), and not compete with introduced thioesterase activity (FIG. 1). Cyanobacterial AD removes one carbon moiety from the fatty aldehyde to release alkane and formate (see for example the articles of Schirmer, et al. titled “Microbial biosynthesis of alkanes”, published in Science 329 (5991), pages 559-562 (2010) and Warui et al., titled “Detection of formate, rather than carbon monoxide, as the stoichiometric co-product in conversion of fatty aldehydes to alkanes by a cyanobacterial aldehyde decarbonylase”, published in J. Am. Chem. Soc. 133 (10), pages 3316-3319 (2011) herein incorporated by reference) whilst the FAR complex normally provides fatty aldehyde substrate for bacterial luciferase through the concerted action of fatty acyl transferase (LuxD), fatty acid reductase (LuxC) and fatty aldehyde synthetase (LuxE) (see also the article of Meighen as mentioned above and herein incorporated by reference). To test the hypothesis, the inventors prepared a codon optimised operon consisting of luxC, luxE and luxD from Photorhabdus luminescens situated in multiple cloning site (MCS) 1 of the pACYCDuet-1 vector for expression in E. coli, as described above. The P. luminescens luciferase system was chosen as it possessed a greater temperature range (active up to 45° C.) and greater activity than luciferase from Vibrio fischeri and V harveyi (see also the articles of Westerlund-Karlsson et al., titled “Generation of thermostable monomeric luciferases from Photorhabdus luminescens”, published in Biochem. Biophys. Res. Commun. 296 (5), pages 1072-1076 (2002) and Winson, M. K. et al., titled “Engineering the luxCDABE genes from Photorhabdus luminescens to provide a bioluminescent reporter for constitutive and promoter probe plasmids and mini-Tn5 constructs”, published in FEMS Microbiol. Lett. 163 (2), pages 193-202 (1998) herein incorporated by reference). The gene for AD from N. punctiforme (referred to as NpAD) was inserted into MCS2 of pACYCDuet-1 to create a vector suitable for expression of FAR and AD under control of IPTG-inducible T7 promoters.

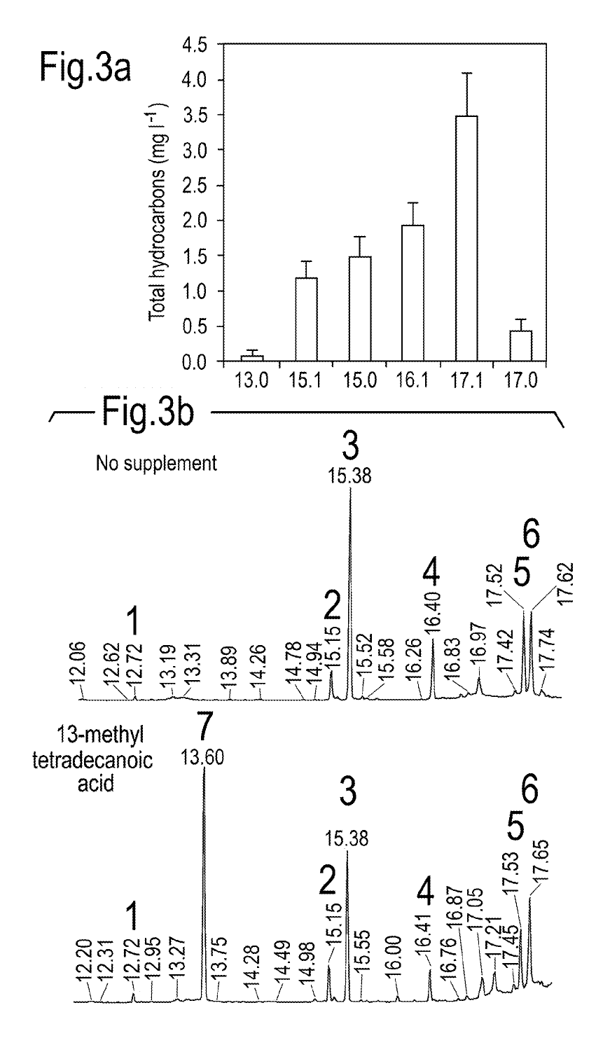

The results indicated that FAR activity was indeed capable of providing substrate for cyanobacterial AD (FIG. 3). The hydrocarbons tridecane, pentadecane, pentadecene, hexadecane and both heptadecane and heptadecane were detected, demonstrating that this engineered pathway produced a range of hydrocarbons of different chain length in vivo. The ability of the new construct to incorporate free FA into alkane biosynthesis was tested by supplementing the growth media with the branched-chain FA 13-methyl tetradecanoic acid, which is not normally present in E. coli. Addition of 13-methyl tetradecanoic acid resulted in the production of branched-chain tridecane (FIG. 3b) demonstrating that the novel pathway possessed the capacity to utilise the free FA pool. Importantly, this result also demonstrated that branched-chain substrates can be metabolised.

In order to test the importance of the fatty acyl transferase to this pathway, luxD was removed from the luxCED operon, to express only luxC, luxE and NpAD. This resulted in an almost complete loss of alkane production that could not be overcome with the addition of the exogenous fatty acids 13-methyl tetradecanoic acid, tetradecanoic acid or hexadecanoic acid. This indicates that for the FAR complex to supply fatty aldehyde to AD, all three LuxCED components are required, though LuxD may not be fulfilling a catalytic role (see also the article of Li, et al. titled “Hyperactivity and interactions of a chimeric myristoyl-ACP thioesterase from the lux system of luminescent bacteria”, published in Biochimica et biophysica acta—protein structure and molecular enzymology 1481 (2), pages 237-246 (2000) herein incorporated by reference).

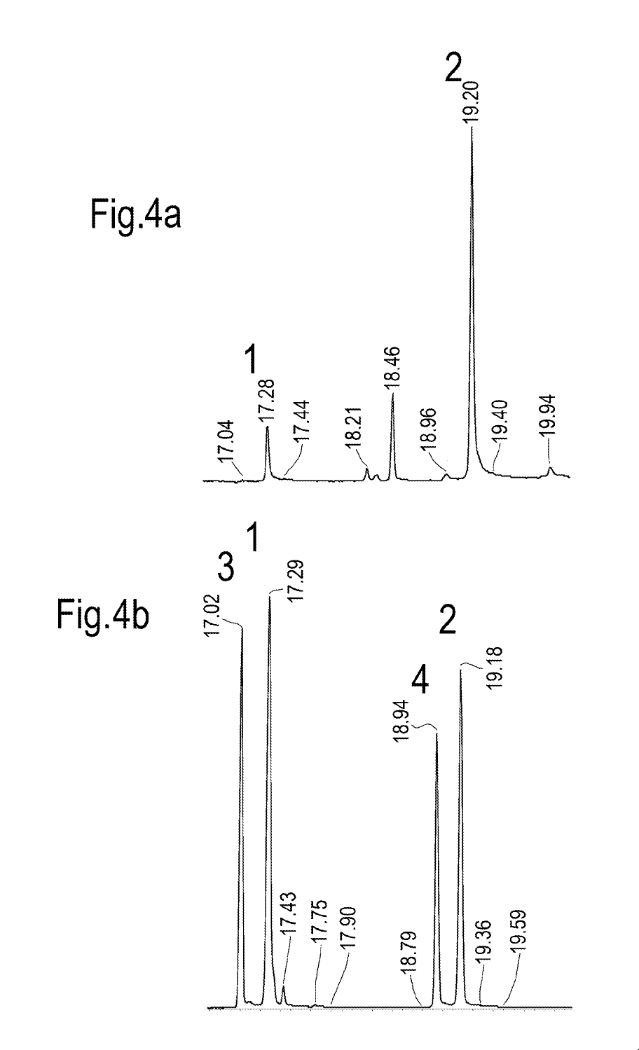

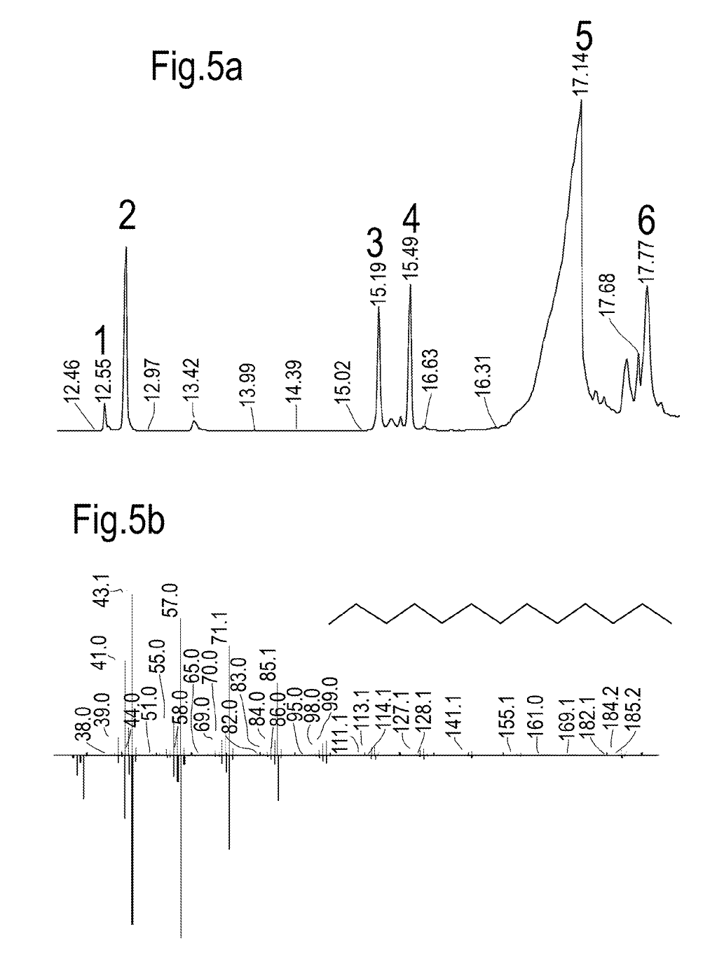

Having established that the FAR-NpAD pathway could incorporate free FAs into alkanes, the inventors sought to achieve the first aim by producing fuel-grade chain length alkanes by modifying the free FA substrate pool. Expression of a cDNA encoding the thioesterase FatB1 from Cinnamomum camphora (camphor) in E. coli leads to the accumulation of tetradecanoic acid (see also the article of Yuan et al. titled “Modification of the substrate specificity of an acyl-acyl carrier protein thioesterase by protein engineering”, published in Proc. Natl. Acad. Sci. U.S.A. 92 (23), pages 10639-10643 (1995) herein incorporated by reference). Such a modification, in combination with the FAR-NpAD pathway, was proposed to alter the final alkane chain length (FIG. 1). A codon-optimised gene encoding FatB1 from C. camphora was inserted into the pETDuet-1 vector and expression in E. coli resulted in the accumulation of tetradecanoic acid (C14) (FIG. 4). Co-expression with the FAR-NpAD pathway gave rise to E. coli cells which exclusively produced tridecane, rather than a range of hydrocarbon chain lengths (FIG. 5). Thus it is possible to manipulate E. coli FA metabolism to the sole production of hydrocarbons that are suitable as next generation biofuel supplements.

The second challenge was to demonstrate the feasibility of producing branched-chain as well as linear hydrocarbons. Branched hydrocarbons are of crucial importance in the performance of fuels at low temperature and high altitude. Given that branched-chain alkanes could be produced when branched-chain FAs were present in the media (FIG. 3b) it was reasoned that it was necessary to generate a branched-chain FA pool in E. coli. E. coli however, produces exclusively straight chain FA. This is because the endogenous 3-ketoacyl-ACP synthase III (KASIII) enzyme (encoded by fabH) only accepts linear acetyl-CoA or propionyl-CoA substrates (FIG. 1). Many Gram-positive bacteria do however produce branched chain FAs (see for example the articles of Kaneda titled “Iso-fatty and anteiso-fatty acids in bacteria—biosynthesis, function, and taxonomic Significance”, published in Microbiol. Rev. 55 (2), pages 288-302 (1991) and Smirnova et al., titled “Branched-chain fatty acid biosynthesis in Escherichia coli”, published in J. Ind. Microbiol. Biotechnol. 27 (4), pages 246-251 (2001) herein incorporated by reference) and, moreover, branched-chain FAs can be produced by E. coli FA elongation enzymes in vitro if an alternative KASIII enzyme (from Bacillus subtilis) and suitable precursor molecules are present (see for example the articles of Smirnova et al., titled “Branched-chain fatty acid biosynthesis in Escherichia coli”, published in J. Ind. Microbiol. Biotechnol. 27 (4), pages 246-251 (2001) and Choi, et al., titled “beta-ketoacyl-acyl carrier protein synthase III (FabH) is a determining factor in branched-chain fatty acid biosynthesis”, published in J. Bacteriol. 182 (2), pages 365-370 (2000) herein incorporated by reference). Expression of B. subtilis KASIII (BsFabH1 or BsFabH2) in E. coli is not enough to achieve this, because the branched biosynthetic primers (keto-acyl CoAs) are lacking (see also the article of Smirnova et al. titled “Branched-chain fatty acid biosynthesis in Escherichia coli”, published in J. Ind. Microbiol. Biotechnol. 27 (4), pages 246-251 (2001) herein incorporated by reference.

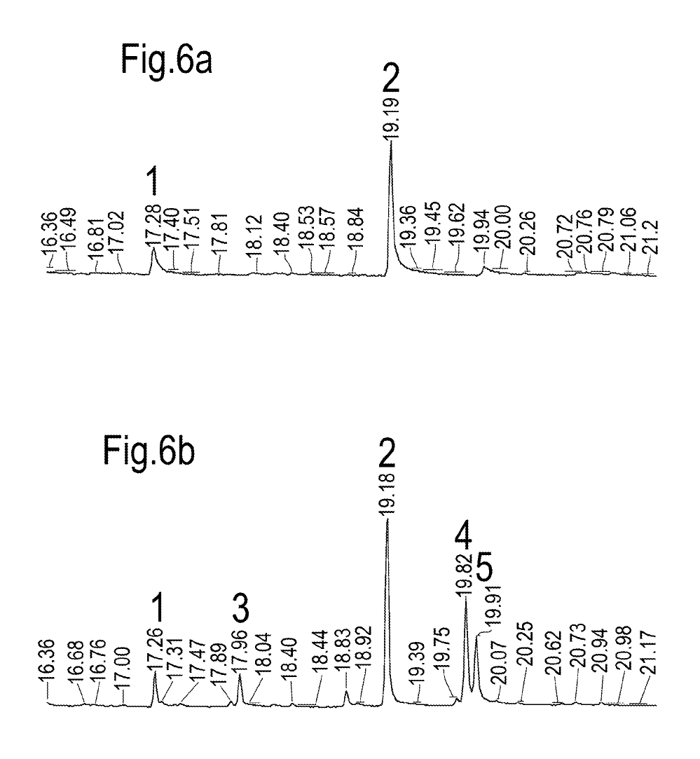

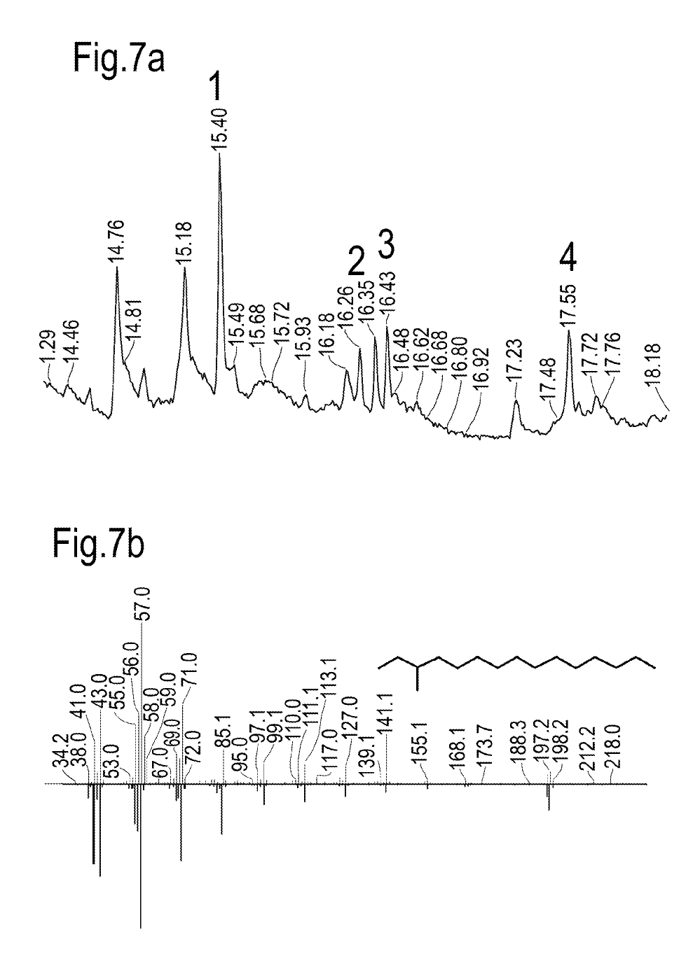

To overcome this limitation the inventors' approach was to supply substrates for B. subtilis KASIII through the introduction of branched-chain keto dehydrogenase (BCKD) activity. The BCKD complex is a multi-enzyme protein complex catalysing three reactions and comprising four subunits: E1α, E1β, E2 and E3 (see for example the articles of Kaneda, titled “Biosynthesis of branched long-chain fatty acids from related short-chain alpha keto acid substrates by a cell-free system of Bacillus subtilis”, published in Can. J. Microbiol. 19 (1), pages 87-96 (1973); Oku, et al., titled “Biosynthesis of branched-chain fatty acids in Bacillis subtilis—a decarboxylase is essential for branched-chain fatty acid synthetase”, published in J. Biol. Chem. 263 (34), pages 18386-18396 (1988); and Skinner et al., titled “Cloning and sequencing of a cluster of genes encoding branched-chain alpha-keto aid dehydrogenase from Streptomyces avermitilis and the production of a functional E1 [alpha beta] component in Escherichia coli”, published in J. Bacteriol. 177 (1), pages 183-190 (1995) herein incorporated by reference). The BCKD complex converts keto acids to keto acyl-CoA in a two step process catalysed by the E1 and E2 subunits whilst the E3 subunit is required for recycling of the lipoamide-E co-factor. When designing the metabolic pathway the inventors reasoned that the substrates for the BCKD complex may be supplied through the endogenous activity of branched chain amino acid aminotransferase (E.C. 2.6.1.42) using the branched amino acids isoleucine, leucine and valine as its substrates (FIG. 1). To test the hypothesis, a codon-optimised five-gene operon in MCS1 of the pETDuet-1 vector was constructed, encoding the four genes required for the B. subtilis BCKD complex, plus fabHB (encoding the B. subtilis KASIII enzyme FabH2). Expression of BCKD and B. subtilis FabH2 in E. coli cells resulted in the production of the branched FAs 13-methyl tetradecanoic acid and 15-methyl hexadecanoic acid (FIG. 6). To test whether the alkane biosynthetic pathway and branched FA pathway could operate in the same cells, all nine genes (five for branched chain fatty acids and four for alkane production) were expressed simultaneously. Co-expression resulted in the production of branched chain pentadecane (FIG. 7) and, therefore, it has been shown that it is possible to synthesise branched alkanes in a microbial host.

The search for sustainable energy sources to mitigate and eventually replace dependence on fossil hydrocarbons may be a priority for the 21^{st} century. The major challenge may be to produce these compounds in bulk and at a cost competitive with, or cheaper than current fuel production costs (see for example the article of Khalil et al., titled “Synthetic biology: applications come of age” published in Nat Rev Genet. 11 (5), pages 367-379 (2010) herein incorporated by reference). Effective deployment of such innovations into the market may ensure rapid adoption of new, sustainable biofuels that benefit the environment and the many consumers that rely on a steady supply of high quality transport fuel. Costs may be reduced further if cheaper source materials could be metabolized. An example of a route to achieving this was recently described by Bokinsky, G. et al., in their article titled “Synthesis of three advanced biofuels from ionic liquid-pretreated switchgrass using engineered Escherichia coli.”, published in Proceedings of the National Academy of Sciences (2011) (herein incorporated by reference). Bokinsky et al described that by the engineering of E. coli it can be made capable of digesting pre-treated lignocellulosic material. Such advances in synthetic biology may have a genuine and lasting impact on the fuel market. The results presented below demonstrate the concept of metabolically derived, renewable, next generation hydrocarbons.

### EXAMPLES

### Expression of Recombinant Enzymes in E. Coli (Bacterial Culture)

Gene expression was under control of the Isopropyl-β-D-1-thiogalactopyranoside(IPTG)-inducible T7 promoter.

The vectors used included pACYCDuet-1, pCDFDuet-1 and pETDuet-1 (all commercially available from Merck Millipore as Novagen Duet vectors). The pACYCDuet-1 vector carries the P15A replicon, lacI gene and chloramphenicol resistance gene; the pCDFDuet-1 vector carries the CloDF13 replicon, lacI gene and streptomycin/spectinomycin resistance gene (aadA); and the pETDuet-1 vector carries the pBR322-derived ColE1 replicon, lacI gene and ampicillin resistance gene.

E. coli BL21(DE3) competent cells * (commercially obtainable from Promega, U.K.) were transformed as follows, using the heat-shock protocols as described by the manufacturer's protocol “INSTRUCTIONS FOR USE OF PRODUCTS L1001, L1191, L2001 AND L2011” unless indicated otherwise: The E. coli cells (stored in sterile polypropylene culture tubes) were removed from the freezer (kept at −80° C.) and chilled on ice. The frozen competent cells were thawed on ice. Subsequently the thawed competent cells were gently mixed by flicking the tube. About 1-50 nanograms (ng) of vector DNA was adjusted with water to 0.5-2 microliters (μl) volume and was mixed gently with the competent cells in each respective tube. Hereafter the tubes were immediately returned to ice for at least 30 minutes. The cells were heat-shocked for 30 seconds in a water bath at about 42° C., without shaking. Subsequently the tubes were immediately placed on ice for 2 minutes. 250 microliters (μl) of warm (37° C.) Super Optimal broth with Catabolite repression (SOC) medium were added to each transformation reaction, followed by incubation for 60 minutes at 37° C. with shaking. 20 μl of the undiluted cells (although also optionally 50 or 100 μl of cells may be used) were plated onto agar plates containing chloramphenicol antibiotic (for pACYCDuet-1), respectively streptomycin/spectinomycin antibiotic (for pCDFDuet-1), respectively ampicillin antibiotic (for pETDuet-1). The plates were incubated at 37° C. for about 12-14 hours or overnight.

A single colony harbouring the relevant plasmid was transferred into a 4 milliliter (ml) Lysogeny broth (LB) medium supplemented with respective antibiotic(s) as mentioned above for selection and the culture was incubated overnight at 37° C., 180 rpm. 50 ml of MMM (modified minimal medium) having the following composition:

### 6 g/l Na2HPO4,

### 3 g/l KH2PO4,

### 0.5 g/l NaCl,

### 2 g/l NH4Cl,

### 0.25 g/l MgSO4×7 H2O,

11 mg/l CaCl2,
27 mg/l Fe3Cl×6 H2O,
2 mg/l ZnCl×4 H2O,
2 mg/l Na2MoO4×2 H2O,
1.9 mg/l CuSO4×5 H2O,
0.5 mg/l H3BO3,
1 mg/l thiamine,
200 mM bis(tris(hydroxymethyl)methylamino)propane (Bis-Tris) (pH 7.25),
0.1% (v/v) Triton-X100 (commercially obtainable from Sigma) and 3% glucose as carbon source.
supplemented with the respective antibiotic(s) as mentioned above and 0.5 grams/liter (g/l) yeast extract (referred to as minimal yeast extract MYE) was inoculated with 500 μl of the overnight culture and incubated at 37° C., 180 rpm until the culture reached an OD600 of 0.6-0.7 unless otherwise indicated. Protein expression was induced by the addition of 20 μM IPTG.

### Hydrocarbon Extraction and Detection

8 ml of bacterial culture was mixed with 8 ml of ethyl acetate and incubated for 2 hours at room temperature (about 20° C.) and 480 rpm. After extraction, samples were centrifuged at room temperature (about 20° C.), 700×gravitation for 5 minutes to cause phase separation and 6 ml of the top phase was transferred into a fresh vial. The ethyl acetate was dried under a stream of nitrogen and subsequently the residue dissolved in 225 ml dichloromethane (DCM). Separation and identification of hydrocarbons and volatile compounds was performed using a Trace GasChromatography-Mass spectrometer (GC/MS) 2000 (Thermo Finnigan) equipped with a ZB1-MS column (commercially obtainable from Zebron). After splitless injection, temperature was kept at 35 C for 2 min and was then increased to 320 C at a rate of 10 C/minute with a subsequent incubation at 320 C for 5 minutes. Injector temperature was kept at 250 C and the flow rate of the carrier gas was 1.0 ml/minute. Scan range of the mass spectrometer was 30-700 m/z at a scan rate of 1.6 scans/second.

For example, FIGS. 2, 3, 5 and 7 illustrate that hydrocarbons such as for example methyl-pentadecane, heptadecene, heptadecane, pentadecane, tridecane, pentadecene, hexadecene, heptadecene, heptadecane, tridecene, methyl-pentadecane can be prepared with the methods of the embodiments of the invention.

Further FIG. 5, for example illustrates that fatty aldehydes (that can be used as fatty aldehyde hydrocarbon precursors) such as trans-5-dodecanal or tetradecanal can be prepared with the methods of the current invention.

### Fatty Acid Extraction and Detection

In order to extract fatty acids, lipids from wet cell pellets and culture supernatants were extracted with dichloromethane (DCM) and methanol (in a DCM:methanol volume ratio of 2:1) according to the Folch method. Fatty acids dissolved in 150 μl DCM were derivatised using 15 μl BSTFA (bis(trimethylsilyl)trifluoroacetamide), commercially obtainable from Supelco Analytical, USA)—whilst ensuring that sufficient BSTFA is used to achieve full derivatisation, at 70° C. during about 1 to 2 hours and analysed using the same programme for hydrocarbon extraction and detection mentioned above.

### Construction of FAS/NpAD Plasmids

The amino acid sequences listed in Table 2 below were reverse translated and codon-optimised for expression in E. coli, providing the nucleic acid sequences also shown in Table 2:

TABLE 2

| | | | Codon-optimised |
|---|---|---|---|
| GenBank** | | | nucleic acid |
| accession number | Sequence name | SEQ ID NO | SEQ ID NO |
| AAD05355.1 | Fatty acid reductase | 1 | 11 |
| AAD05359.1 | LuxE E | 2 | 12 |
| P19197.1 | LUXD1_PHOLU | 3 | 13 |

**The sequences can be retrieved from GenBank at http://www.ncbi.nlm.nih.gov/genbank.

GenBank is the NIH genetic sequence database. Genbank is located at the National Center for Biotechnology Information, U.S. National Library of Medicine, 8600 Rockville Pike, Bethesda Md., 20894 USA.

Codon-optimised luxC, luxE and luxD genes for E. Coli were synthesised in a three-gene operon (SEQ ID NO:15) inserted into pACYCDuet-1 (commercially obtainable from Merck, the final construct having sequence SEQ ID NO:16) and subsequently digested with the restriction enzymes NcoI and NotI (commercially obtainable) and ligated into pCDFDuet-1 MCS1 (commercially obtainable from Merck).

The Genomic DNA was extracted from N. punctiforme using the FAST-DNA SPIN Kit (commercially obtainable by MP Biomedicals). Cultures were centrifuged for 2 min, 4500 rpm, 4 C and 120 mg of the pellet was re-suspended in 1 ml of buffer Cell Lysis/DNA Solubilizing Solution (CLS-Y). Samples were homogenized with a MP Biomedicals FastPrep-24 (FASTPREP is a trademark) instrument using lysing matrix A (also MP Biomedicals) for 40 sec at a speed setting of 6.0 m/s. All subsequent steps were carried out according to the manufacturer's instructions. After this procedure, the genomic DNA was further purified by phenol-chloroform extraction (using a tris(hydroxymethyl)aminomethane) pH7.5-buffered 50% phenol, 48% chloroform, 2% isoamyl alcohol solution), followed by DNA precipitation using ethanol and sodium acetate. The final DNA samples were adjusted (using water) to a concentration of 8 nanograms per microliter (ng/μl). The gene encoding NpAD (aldehyde decarbonylase) was amplified with PHUSION High-Fidelity DNA Polymerase (PHUSION is a trademark, commercially obtainable from New England Biolabs), using 8 ng of cyanobacterial genomic DNA as template.

Primers used were CATATGCAGCAGCTTACAGACCAAT (SEQ ID NO:26) and CTCGAGTTAAGCACCTATGAGTCCGTAGG (SEQ ID NO:27), allowing direct cloning into MCS2 (MCS is an abbreviation for Multiple Cloning Site) using NdeI and XhoI sites (underlined).

Plasmids were transformed into TOP10 competent E. coli cells (commercially obtainable from Invitrogen) using the manufactures protocol (as described above for Expression of recombinant enzymes in E. coli), purified using the Qiagen miniprep kit (purified plasmids) and insertions were investigated by polymerase chain reaction (PCR) or restriction digest. The nucleic acid sequence SEQ ID NO:13, encoding NpAD, was confirmed to be present in pACYCDuet-1 luxCED and pCDFDuet-1 luxCED by DNA sequencing (commercially obtainable from Geneservice, U.K.) of purified plasmids.

### Construction of Thioesterase Expression Plasmid

The amino acid sequence SEQ ID NO:5 was reverse translated and codon-optimised for expression in E. coli. The gene sequence was digested with NcoI and BamHI and ligated into MCS1 of pETDuet-1, to form SEQ ID NO:18.

### Construction of B. Subtilis BCKD/KASIII Operon Expression Plasmid

The amino acid sequences shown in Table 3 below were reverse translated and codon-optimised for expression in E. coli, providing the nucleic acid sequences also shown in the Table.

TABLE 3

| | | | Codon-optimised |
|---|---|---|---|
| GenBank** | | SEQ ID | nucleic acid |
| accession number | Sequence name | NO | SEQ ID NO |
| NP_388898.1 | 3-ketoacyl-ACP | 6 | 19 |
| | synthase III | | |
| NP_390285.1 | Branched chain alpha- | 7 | 20 |
| | keto acid | | |
| | dehydrogenase E1 | | |
| | subunit | | |
| NP_390284.1 | Branched chain alpha- | 8 | 21 |
| | keto acid | | |
| | dehydrogenase E1 | | |
| | subunit | | |
| NP_390283.1 | Branched chain alpha- | 9 | 22 |
| | keto acid | | |
| | dehydrogenase E2 | | |
| | subunit | | |
| ZP_03600867.1 | Dihydrolipoamide | 10 | 23 |
| | dehydrogenase | | |
| | (Branched chain alpha- | | |
| | keto acid | | |
| | dehydrogenase E3 | | |
| | subunit) | | |

**The sequences can be retrieved from GenBank at http://www.ncbi.nlm.nih.gov/genbank.

GenBank is the NIH genetic sequence database. Genbank is located at the National Center for Biotechnology Information, U.S. National Library of Medicine, 8600 Rockville Pike, Bethesda Md., 20894 USA

The codon-optimised Branched-Chain Keto Dehydrogenase (BCKD) and 3-ketoacyl-ACP synthase III (KASIII), Fatty acid biosynthesis H2 (FabH2) sequences were synthesised in a five-gene operon (SEQ ID NO:24) and digested with NcoI and NotI and ligated into pETDuet-1 MCS1 (final construct having sequence SEQ ID NO:25).

### CONCLUSION

In conclusion, the results presented here demonstrate one exemplary embodiment providing de novo construction of a synthetic metabolic pathway for custom alkane biosynthesis capable of utilising the cellular free FA pool directly. The inventors have shown that by modifying the free FA pool it is possible to produce both fuel-grade and branched alkanes in a bacterial host from basic, renewable ingredients.

In Table 4 below the identity of sequences included in this specification is provided.

TABLE 4
Identity of sequences included in application

| SEQ ID NO | Description of sequence |
|---|---|
| 1 | Photorhabdus luminescens LuxC amino acid sequence |
| 2 | P. luminescens LuxE amino acid sequence |
| 3 | P. luminescens LuxD amino acid sequence |
| 4 | Nostoc punctiforme aldehyde decarbonylase |
| | amino acid sequence |
| 5 | Cinnamomum camphora thioesterase amino acid sequence |
| 6 | Bacillus subtilis KasIII (3-ketoacyl-ACP synthase III) |
| | amino acid sequence |
| 7 | B. subtilis BCKD subunit E1α amino acid sequence |
| 8 | B. subtilis BCKD subunit E1β amino acid sequence |
| 9 | B. subtilis BCKD subunit E2 amino acid sequence |
| 10 | B. subtilis BCKD subunit E3 amino acid sequence |
| 11 | P. luminescens LuxC codon-optimised nucleotide sequence |
| 12 | P. luminescens LuxE codon-optimised nucleotide sequence |
| 13 | N. punctiforme aldehyde decarbonylase codon-optimised |
| | nucleotide sequence |
| 14 | P. luminescens LuxD codon-optimised nucleotide sequence |
| 15 | P. luminescens LuxCDE operon codon-optimised |
| | nucleotide sequence |
| 16 | pACYC LuxCDE |
| 17 | C. camphora thioesterase codon-optimised nucleotide |
| | sequence |
| 18 | pETDuet-1 thioesterase |
| 19 | B. subtilis KasIII codon-optimised nucleotide sequence |
| 20 | B. subtilis BCKD subunit E1α codon-optimised |
| | nucleotide sequence |
| 21 | B. subtilis BCKD subunit E1β codon-optimised |
| | nucleotide sequence |
| 22 | B. subtilis BCKD subunit E2 codon-optimised nucleotide |
| | sequence |
| 23 | B. subtilis BCKD subunit E3 codon-optimised nucleotide |
| | sequence |
| 24 | KasIII/BCKD operon codon-optimised nucleotide sequence |
| 25 | pETDuet-1 KasIII/BCKD |
| 26 | Amplification primer |
| 27 | Amplification primer |
| 28 | Vibrio harveyi LuxC amino acid sequence |
| 29 | Vibrio fischeri ES114 LuxC amino acid sequence |
| 30 | Vibrio harveyi LuxD amino acid sequence |
| 31 | Vibrio fischeri MJ11 LuxD amino acid sequence |
| 32 | Vibrio harveyi LuxE amino acid sequence |
| 33 | Vibrio fischeri ES114 LuxE amino acid sequence |

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as the presently preferred embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

What is claimed is:

1. A method for preparing a hydrocarbon comprising:
obtaining a fatty acid aldehyde by contacting a fatty acid substrate with at least one fatty acid reductase, at least one fatty aldehyde synthetase, and at least one fatty acyl transferase, wherein the fatty acid substrate is selected from the group consisting of a fatty acid, a fatty acyl-ACP, a fatty acyl-CoA and any combination thereof; and
obtaining a hydrocarbon by contacting the fatty aldehyde with at least one aldehyde decarbonylase.

2. The method of claim 1, wherein the fatty acid reductase is a polypeptide in class EC 1.2.1.50.

3. The method of claim 2, wherein the fatty acid reductase is a polypeptide comprising
an amino acid sequence at least 50% identical to SEQ ID NO:1.

4. The method of claim 3, wherein the amino acid sequence is selected from the group consisting of SEQ ID NO:1, SEQ ID NO:28, and SEQ ID NO:29.

5. The method of claim 1, wherein the fatty aldehyde synthetase is a polypeptide in class EC 6.2.1.19.

6. The method of claim 5, wherein the fatty aldehyde synthetase is a polypeptide comprising an amino acid sequence at least 50% identical to SEQ ID NO:2.

7. The method of claim 6, wherein the amino acid sequence is selected from the group consisting of SEQ ID NO:2, SEQ ID NO:32, and SEQ ID NO:33.

8. The method of claim 1, wherein the fatty acyl transferase is a polypeptide in class EC 2.3.1.

9. The method of claim 8, wherein the fatty acyl transferase is a polypeptide comprising an amino acid sequence at least 50% identical to SEQ ID NO:3.

10. The method of claim 9, wherein the amino acid sequence is selected from the group consisting of SEQ ID NO:3, SEQ ID NO:30, and SEQ ID NO:31.

11. The method of claim 1, wherein the aldehyde decarbonylase is a polypeptide in class EC 4.1.99.5.

12. The method of claim 11, wherein the aldehyde decarbonylase is a polypeptide comprising an amino acid sequence at least 50% identical to SEQ ID NO:4.

13. The method of claim 1, further comprising obtaining at least a portion of said fatty acid substrate by contacting a fatty acyl-ACP with at least one acyl-ACP thioesterase.

14. The method of claim 13, wherein the acyl-ACP thioesterase is a polypeptide in class EC 3.1.2.14.

15. The method of claim 14, wherein the acyl-ACP thioesterase is a polypeptide comprising an amino acid sequence at least 50% identical to SEQ ID NO:5.

16. The method of claim 1, wherein at least some of said fatty acid substrate is a fatty acyl-ACP, said method further comprising obtaining at least a portion of said fatty acyl-ACP by contacting a keto-acyl-CoA and a malonyl-ACP with at least one 3-ketoacyl-ACP synthase III.

17. The method of claim 16, wherein the 3-ketoacyl-ACP synthase III is a polypeptide in class EC 2.3.1.180.

18. The method of claim 17, wherein the 3-ketoacyl-ACP synthase III is a polypeptide comprising an amino acid sequence at least 75% identical to SEQ ID NO:6.

19. The method of claim 16, further comprising obtaining at least a portion of the keto acyl-CoA by contacting a keto acid with a branched-chain ketodehydrogenase complex.

20. The method of claim 19, wherein the branched-chain ketodehydrogenase complex comprises a polypeptide in class EC 1.2.4.4 and a polypeptide in class EC 2.3.1.168 and a polypeptide in class 1.8.1.4.

21. The method of claim 20, wherein the branched-chain ketodehydrogenase complex comprises a polypeptide comprising an amino acid sequence at least 50% identical to SEQ ID NO:7, a polypeptide comprising an amino acid sequence at least 50% identical to SEQ ID NO:8, a polypeptide comprising an amino acid sequence at least 50% identical to SEQ ID NO:9, a polyeptide comprising an amino acid sequence at least 50% identical to SEQ ID NO:10.

22. The method of claim 1, wherein the hydrocarbon is selected from: a straight chain alkane, a straight chain alkene, a branched chain alkane, a branched chain alkene, and any combination of any two or more thereof.

23. The method of claim 22, wherein the hydrocarbon comprises between 4 and 30 carbon atoms.

24. The method of claim 1, wherein at least one of the fatty acid reductase, the fatty aldehyde synthetase, the fatty acyl transferase, and the aldehyde decarbonylase is expressed by a recombinant host cell.

25. The method of claim 24, wherein the recombinant host cell is a genetically modified microorganism genetically modified to express at least one exogenous polypeptide selected from the group consisting of fatty acid reductase, fatty aldehyde synthetase, aldehyde decarbonylase, and fatty acyl transferase.

26. The method of claim 25, wherein the host cell comprises at least one nucleic acid encoding one or more of the amino acid sequences selected from the group consisting of SEQ ID NO:1 to SEQ ID NO:4.

27. The method of claim 24, wherein the recombinant host cell is a yeast or a bacterium.

28. The method of claim 27, wherein the yeast is Saccharomyces cerevisiae and the bacterium is Eschericia coli.

29. The method of claim 13, wherein the acyl-ACP thioesterase is expressed by a recombinant host cell.

30. The method of claim 29, wherein the recombinant host cell is a genetically modified microorganism genetically modified to express an exogenous acyl-ACP thioesterase.

31. The method of claim 30, wherein the host cell comprises at least one nucleic acid encoding the amino acid sequence of SEQ ID NO:5.

32. The method of claim 29, wherein the recombinant host cell is a yeast or a bacterium.

33. The method of claim 32, wherein the yeast is Saccharomyces cerevisiae and the bacterium is Eschericia coli.

34. The method of claim 16, wherein the 3-ketoacyl-ACP synthase III is expressed by a recombinant host cell.

35. The method of claim 34, wherein the recombinant host cell is a genetically modified microorganism genetically modified to express an exogenous 3-ketoacyl-ACP synthase III.

36. The method of claim 35, wherein the host cell comprises at least one nucleic acid encoding the amino acid sequence of SEQ ID NO:6.

37. The method of claim 34, wherein the recombinant host cell is a yeast or a bacterium.

38. The method of claim 37, wherein the yeast is Saccharomyces cerevisiae and the bacterium is Eschericia coli.

39. The method of claim 19, wherein the branched-chain ketodehydrogenase complex is expressed by a recombinant host cell.

40. The method of claim 39, wherein the recombinant host cell is a genetically modified microorganism genetically modified to express an exogenous branched-chain ketodehydrogenase complex.

41. The method of claim 40, wherein the host cell comprises at least one nucleic acid encoding one or more of the amino acid sequences selected from the group consisting of SEQ ID NO:7 to SEQ ID NO:10.

42. The method of claim 39, wherein the recombinant host cell is a yeast or a bacterium.

43. The method of claim 42, wherein the yeast is Saccharomyces cerevisiae and the bacterium is Eschericia coli.

44. A recombinant host cell comprising a fatty acid reductase and a fatty aldehyde synthetase and a fatty acyl transferase.

45. The recombinant host cell of claim 44, further comprising an aldehyde decarbonylase.

46. The recombinant host cell of claim 44 comprising a polypeptide in class EC 1.2.1.50, a polypeptide in class EC 6.2.1.19, a polypeptide in class EC 2.3.1.

47. The recombinant host cell of claim 45 comprising a polypeptide in class EC 4.1.99.5.

48. The recombinant host cell of claim 47 comprising a polypeptide comprising an amino acid sequence at least 50% identical to SEQ ID NO:1; a polypeptide comprising an amino acid sequence at least 50% identical to SEQ ID NO:2; a polypeptide comprising an amino acid sequence at least 50% identical to SEQ ID NO:3; a polypeptide comprising an amino acid sequence at least 50% identical to SEQ ID NO:4.

49. The recombinant host cell of claim 48, wherein the amino acid sequence at least 50% identical to SEQ ID NO:1 is selected from the group consisting of SEQ ID NO:1, SEQ ID NO:28, and SEQ ID NO:29; the amino acid sequence at least 50% identical to SEQ ID NO:2 is selected from the group consisting of SEQ ID NO:2, SEQ ID NO:32, and SEQ ID NO:33; the amino acid sequence at least 50% identical to SEQ ID NO:3 is selected from the group consisting of SEQ ID NO:3, SEQ ID NO:30, and SEQ ID NO:31.

50. The recombinant host cell of claim 48 further comprising a polynucleotide comprising at least one sequence selected the group consisting of SEQ ID NO:11 to SEQ ID NO:16.

51. The recombinant host cell of claim 44 further comprising a polypeptide in class EC 3.1.2.14.

52. The recombinant host cell of claim 51 comprising a polypeptide comprising an amino acid sequence at least 50% identical to SEQ ID NO:5.

53. The recombinant host cell of claim 52 further comprising a polynucleotide have a nucleotide sequence selected from the group consisting of SEQ ID NO:17 and SEQ ID NO:18.

54. The recombinant host cell of claim 52 further comprising a polypeptide in class EC 2.3.1.180.

55. The recombinant host cell of claim 54 comprising an amino acid sequence at least 50% identical to SEQ ID NO:6.

56. The recombinant host cell of claim 55 further comprising a polynucleotide comprising nucleotide sequence of SEQ ID NO:19.

57. The recombinant host cell of claim 54 further comprising a polypeptide in class EC 1.2.4.4, a polypeptide in class EC 2.3.1.168, a polypeptide in class EC 1.8.1.4.

58. The recombinant host cell of claim 57 comprising a polypeptide comprising an amino acid sequence at least 50% identical to SEQ ID NO:7; a polypeptide an amino acid sequence at least 50% identical to SEQ ID NO:8; a polypeptide comprising an amino acid sequence at least 50% identical to SEQ ID NO:9; and a polypeptide comprising an amino acid sequence at least 50% identical to SEQ ID NO:10.

59. The recombinant host cell of claim 58 further comprising a polynucleotide comprising a sequence selected from the group consisting of SEQ ID NO:20 to SEQ ID NO:23.

60. The recombinant host cell of claim 59 further comprising a polynucleotide comprising a sequence selected from the group consisting of SEQ ID NO:24 SEQ ID NO:25.

61. An expression vector comprising at least one polynucleotide comprising a sequence selected from the group consisting of SEQ ID NO:11 to SEQ ID NO:25, a complement thereof, and a sequence which will hybridize under stringent conditions to any such polynucleotide sequence.

62. A method of producing an alkane, comprising hydrogenation of an isolated alkene produced in a method of claim 1.

63. A method of producing a branched alkane, comprising hydroisomerization of an isolated alkene and/or alkane produced in a method of claim 1.

64. A method for the production of a biofuel and/or a biochemical comprising combining an alkene and/or alkane produced in a method of claim 1 or an alkane produced in a method according to claim 62 with one or more additional components to produce a biofuel and/or biochemical.


SEQUENCE LISTING (33 sequences)

SEQ ID NO: 1 - protein, 480 aa, Photorhabdus luminescens
MNKKISFIINGRVEIFPESDDLVQSINFGDNSVHLPVLNDSQVKNIIDYNENNELQLHNIINFLYTVGQRWKNEEYSRRRTYIRDLKRYMGYSEEMAKLEANWISMILCSKGGLYDLVKNELGSRHIMDEWLPQDESYIRAFPKGKSVHLLTGNVPLSGVLSILRAILTKNQCIIKTSSTDPFTANALALSFIDVDPHHPVTRSLSVVYWQHQGDISLAKEIMQHADVVVAWGGEDAINWAVKHAPPDIDVMKFGPKKSFCIIDNPVDLVSAATGAAHDVCFYDQQACFSTQNIYYMGSHYEEFKLALIEKLNLYAHILPNTKKDFDEKAAYSLVQKECLFAGLKVEVDVHQRWMVIESNAGVELNQPLGRCVYLHHVDNIEQILPYVRKNKTQTISVFPWEAALKYRDLLALKGAERIVEAGMNNIFRVGGAHDGMRPLQRLVTYISHERPSHYTAKDVAVEIEQTRFLEEDKFLVFVP

SEQ ID NO: 2 - protein, 370 aa, Photorhabdus luminescens
MTSYVDKQEITASSEIDDLIFSSDPLVWSYDEQEKIRKKLVLDAFRHHYKHCQEYRHYCQAHKVDDNITEIDDIPVFPTSVFKFTRLLTSNENEIESWFTSSGTNGLKSQVPRDRLSIERLLGSVSYGMKYIGSWFDHQMELVNLGPDRFNAHNIWFKYVMSLVELLYPTSFTVTEEHIDFVQTLNSLERIKHQGKDICLIGSPYFIYLLCRYMKDKNISFSGDKSLYIITGGGWKSYEKESLKRNDFNHLLFDTFNLSNINQIRDIFNQVELNTCFFEDEMQRKHVPPWVYARALDPETLKPVPDGMPGLMSYMDASSTSYPAFIVTDDIGIISREYGQYPGVLVEILRRVNTRKQKGCALSLTEAFGS

SEQ ID NO: 3 - protein, 307 aa, Photorhabdus luminescens
MENESKYKTIDHVICVEGNKKIHVWETLPEENSPKRKNAIIIASGFARRMDHFAGLAEYLSRNGFHVIRYDSLHHVGLSSGTIDEFTMSIGKQSLLAVVDWLTTRKINNFGMLASSLSARIAYASLSEINASFLITAVGFVNLRYSLERALGFDYLSLPINELPNNLDFEGHKLGAEVFARDCLDFGWEDLASTINNMMYLDIPFIAFTANNDNWVKQDEVITLLSNIRSNRCKIYSLLGSSHDLSENLVVLRNFYQSVTKAAIAMDNDHLDIDVDITEPSFEHLTIATVNERRMRIEIENQAISLS

SEQ ID NO: 4 - protein, 232 aa, Nostoc punctiforme
MQQLTDQSKELDFKSETYKDAYSRINAIVIEGEQEAHENYITLAQLLPESHDELIRLSKMESRHKKGFEACGRNLAVTPDLQFAKEFFSGLHQNFQTAAAEGKVVTCLLIQSLIIECFAIAAYNIYIPVADDFARKITEGVVKEEYSHLNFGEVWLKEHFAESKAELELANRQNLPIVWKMLNQVEGDAHTMAMEKDALVEDFMIQYGEALSNIGFSTRDIMRLSAYGLIGA

SEQ ID NO: 5 - protein, 300 aa, Cinnamomum camphora
MLEWKPKPNPPQLLDDHFGPHGLVFRRTFAIRSYEVGPDRSTSIVAVMNHLQEAALNHAKSVGILGDGFGTTLEMSKRDLIWVVKRTHVAVERYPAWGDTVEVECWVGASGNNGRRHDFLVRDCKTGEILTRCTSLSVMMNTRTRRLSKIPEEVRGEIGPAFIDNVAVKDEEIKKPQKLNDSTADYIQGGLTPRWNDLDINQHVNNIKYVDWILETVPDSIFESHHISSFTIEYRRECTMDSVLQSLTTVSGGSSEAGLVCEHLLQLEGGSEVLRAKTEWRPKLTDSFRGISVIPAESSV

SEQ ID NO: 6 - protein, 325 aa, Bacillus subtilis
MSKAKITAIGTYAPSRRLTNADLEKIVDTSDEWIVQRTGMRERRIADEHQFTSDLCIEAVKNLKSRYKGTLDDVDMILVATTTSDYAFPSTACRVQEYFGWESTGALDINATCAGLTYGLHLANGLITSGLHQKILVIAGETLSKVTDYTDRTTCVLFGDAAGALLVERDEETPGFLASVQGTSGNGGDILYRAGLRNEINGVQLVGSGKMVQNGREVYKWAARTVPGEFERLLHKAGLSSDDLDWFVPHSANLRMIESICEKTPFPIEKTLTSVEHYGNTSSVSIVLALDLAVKAGKLKKDQIVLLFGFGGGLTYTGLLIKWGM

SEQ ID NO: 7 - protein, 330 aa, Bacillus subtilis
MSTNRHQALGLTDQEAVDMYRTMLLARKIDERMWLLNRSGKIPFVISCQGQEAAQVGAAFALDREMDYVLPYYRDMGVVLAFGMTAKDLMMSGFAKAADPNSGGRQMPGHFGQKKNRIVTGSSPVTTQVPHAVGIALAGRMEKKDIAAFVTFGEGSSNQGDFHEGANFAAVHKLPVIFMCENNKYAISVPYDKQVACENISDRAIGYGMPGVTVNGNDPLEVYQAVKEARERARRGEGPTLIETISYRLTPHSSDDDDSSYRGREEVEEAKKSDPLLTYQAYLKETGLLSDEIEQTMLDEIMAIVNEATDEAENAPYAAPESALDYVYAK

SEQ ID NO: 8 - protein, 327 aa, Bacillus subtilis
MSVMSYIDAINLAMKEEMERDSRVFVLGEDVGRKGGVFKATAGLYEQFGEERVMDTPLAESAIAGVGIGAAMYGMRPIAEMQFADFIMPAVNQIISEAAKIRYRSNNDWSCPIVVRAPYGGGVHGALYHSQSVEAIFANQPGLKIVMPSTPYDAKGLLKAAVRDEDPVLFFEHKRAYRLIKGEVPADDYVLPIGKADVKREGDDITVITYGLCVHFALQAAERLEKDGISAHVVDLRTVYPLDKEAIIEAASKTGKVLLVTEDTKEGSIMSEVAAIISEHCLFDLDAPIKRLAGPDIPAMPYAPTMEKYFMVNPDKVEAAMRELAEF

SEQ ID NO: 9 - protein, 424 aa, Bacillus subtilis
MAIEQMTMPQLGESVTEGTISKWLVAPGDKVNKYDPIAEVMTDKVNAEVPSSFTGTITELVGEEGQTLQVGEMICKIETEGANPAEQKQEQPAASEAAENPVAKSAGAADQPNKKRYSPAVLRLAGEHGIDLDQVTGTGAGGRITRKDIQRLIETGGVQEQNPEELKTAAPAPKSASKPEPKEETSYPASAAGDKEIPVTGVRKAIASNMKRSKTEIPHAWTMMEVDVTNMVAYRNSIKDSFKKTEGFNLTFFAFFVKAVAQALKEFPQMNSMWAGDKIIQKKDINISIAVATEDSLFVPVIKNADEKTIKGIAKDITGLAKKVRDGKLTADDMQGGTFTVNNTGSFGSVQSMGIINYPQAAILQVESIVKRPVVMDNGMIAVRDMVNLCLSLDHRVLDGLVCGRFLGRVKQILESIDEKTSVY

SEQ ID NO: 10 - protein, 474 aa, Bacillus subtilis
MATEYDVVILGGGTGGYVAAIRAAQLGLKTAVVEKEKLGGTCLHKGCIPSKALLRSAEVYRTAREADQFGVETAGVSLNFEKVQQRKQAVVDKLAAGVNHLMKKGKIDVYTGYGRILGPSIFSPLPGTISVERGNGEENDMLIPKQVIIATGSRPRMLPGLEVDGKSVLTSDEALQMEELPQSIIIVGGGVIGIEWASMLHDFGVKVTVIEYADRILPTEDLEISKEMESLLKKKGIQFITGAKVLPDTMTKTSDDISIQAEKDGETVTYSAEKMLVSIGRQANIEGIGLENTDIVTENGMISVNESCQTKESHIYAIGDVIGGLQLAHVASHEGIIAVEHFAGLNPHPLDPTLVPKCIYSSPEAASVGLTEDEAKANGHNVKIGKFPFMAIGKALVYGESDGFVKIVADRDTDDILGVHMIGPHVTDMISEAGLAKVLDATPWEVGQTIHPHPTLSEAIGEAALAADGKAIHF

SEQ ID NO: 11 - DNA, 1443 bp, Photorhabdus luminescens
atgaacaagaaaatcagcttcatcatcaacggtcgcgtagaaatttttccggagtctgatgacctggttcaaagcatcaattttggtgacaatagcgtccacctgccggtgctgaacgatagccaagtgaaaaacattatcgactataatgagaataatgagctgcaactgcacaatatcattaactttctgtataccgtcggtcagcgctggaaaaacgaagaatacagccgtcgtcgtacctatattcgcgatctgaagcgttatatgggctacagcgaggaaatggcgaaactggaagccaattggattagcatgattctgtgctctaaaggtggtttgtacgatctggtgaaaaatgagctgggcagccgtcacattatggacgaatggctgccgcaagacgaaagctacatccgtgccttcccgaaaggcaagagcgttcatctgctgaccggtaatgtcccgctgtcgggcgtgctgtccatcctgcgcgcgattctgaccaagaaccagtgcatcattaagacgagcagcacggatcctttcacggcgaatgcgctggcgctgagcttcatcgacgttgacccacatcacccggtgacccgtagcctgtctgtcgtttattggcagcaccaaggtgacatcagcttggcgaaagagattatgcagcacgccgatgtggtcgttgcctggggtggtgaggatgcaattaactgggcggttaaacacgcaccgccggatatcgacgtcatgaaattcggtccgaaaaagagcttctgcatcattgacaacccggttgacttggttagcgcagcgaccggcgcagcacacgacgtctgtttttacgatcagcaggcatgctttagcacgcagaacatctactacatgggctcccattacgaggagtttaagctggctttgatcgaaaaactgaatctgtatgcacatatcctgcctaacaccaagaaggatttcgacgaaaaggcagcttattccttggtgcaaaaggagtgtctgttcgccggtttgaaagtggaagttgacgttcatcaacgctggatggttattgaatccaatgctggcgttgagctgaaccagccgctgggtcgttgtgtgtacttgcatcacgtggataacatcgagcagattttgccgtatgtgcgtaagaacaaaacccagacgattagcgtgtttccgtgggaggctgcgctgaagtaccgcgatctgctggccctgaaaggcgcggagcgtattgttgaggcgggtatgaataacattttccgtgtgggtggtgcgcacgatggcatgcgtccgctgcaacgcctggtcacttacattagccacgagcgtccgagccattacaccgcgaaggacgtcgcggtcgaaatcgaacagacgcgctttctggaagaggacaagttcctggtgtttgttccataa

SEQ ID NO: 12 - DNA, 1113 bp, Photorhabdus luminescens
atgactagctacgtcgacaaacaggaaatcaccgcgagcagcgagattgacgacctgatcttttccagcgatccgttggtgtggtcctatgatgagcaagaaaagattcgcaagaaactggtcctggatgcgttccgccaccactacaagcactgtcaagagtaccgtcattattgccaagcccataaagtcgacgataacattacggaaattgacgatatcccggttttcccgacctctgttttcaagttcacccgtctgctgacctccaacgagaatgagattgagagctggtttacttcgagcggtaccaatggtctgaaaagccaagtcccgcgtgatcgtctgagcattgaacgtctgctgggcagcgtgagctacggcatgaagtacatcggttcgtggtttgaccatcaaatggagctggttaacttgggtccggatcgctttaatgcccacaacatttggttcaagtacgttatgagcctggttgagctgttgtatccgacgagcttcaccgtgacggaagagcacatcgacttcgtgcagacgctgaacagcctggaacgcattaaacatcagggcaaagacatttgtctgatcggttctccgtatttcatctatctgctgtgccgttacatgaaggacaagaacatcagctttagcggtgacaagagcctgtatatcatcaccggtggcggttggaaaagctacgaaaaagagtccctgaagcgtaatgactttaatcacctgttgttcgatacgttcaatctgagcaacattaaccagatccgtgacatctttaaccaggtcgaactgaatacctgtttctttgaggacgagatgcagcgcaaacacgtcccgccgtgggtatacgcgcgtgcgctggatcctgaaaccttgaaaccggttccagatggcatgcctggtctgatgagctatatggatgctagctctacgagctacccggcatttatcgtgaccgacgatattggtattatcagccgcgagtacggtcaatatccgggcgtgctggttgaaattctgcgtcgtgtgaatacccgcaagcagaaaggctgcgcgttgtctctgacggaggcattcggttcctaa

SEQ ID NO: 13 - DNA, 699 bp, Nostoc punctiforme
atgcagcagcttacagaccaatctaaagaattagatttcaagagcgaaacatacaaagatgcttatagccggattaatgcgatcgtgattgaaggggaacaagaagcccatgaaaattacatcacactagcccaactgctgccagaatctcatgatgaattgattcgcctatccaagatggaaagccgccataagaaaggatttgaagcttgtgggcgcaatttagctgttaccccagatttgcaatttgccaaagagtttttctccggcctacaccaaaattttcaaacagctgccgcagaagggaaagtggttacttgtctgttgattcagtctttaattattgaatgttttgcgatcgcagcatataacatttacatccccgttgccgacgatttcgcccgtaaaattactgaaggagtagttaaagaagaatacagccacctcaattttggagaagtttggttgaaagaacactttgcagaatccaaagctgaacttgaacttgcaaatcgccagaacctacccatcgtctggaaaatgctcaaccaagtagaaggtgatgcccacacaatggcaatggaaaaagatgctttggtagaagacttcatgattcagtatggtgaagcattgagtaacattggtttttcgactcgcgatattatgcgcttgtcagcctacggactcataggtgcttaa

SEQ ID NO: 14 - DNA, 924 bp, Photorhabdus luminescens
atggaaaacgagagcaagtacaaaacgatcgaccacgtaatctgcgtggagggtaacaaaaagattcacgtgtgggagactttgccagaagagaacagcccgaaacgcaaaaacgcaatcattatcgcgagcggtttcgcacgccgcatggatcattttgcgggcctggccgaatacctgagccgtaacggcttccacgttatccgttatgacagcctgcatcacgtcggcctgtcgtctggtaccatcgacgagttcacgatgagcatcggcaagcaaagcctgttggcggttgttgattggctgaccacgcgtaagatcaacaattttggtatgctggcttccagcctgtccgcacgcattgcgtacgcttctctgagcgagattaatgccagctttctgatcaccgccgtgggtttcgtcaatctgcgttatagcctggagcgtgcgctgggtttcgattacttgagcctgccgattaacgagctgccgaataatctggactttgaaggccataagttgggtgcggaggtctttgcgcgtgattgcctggattttggttgggaagatctggcatcgacgattaacaatatgatgtatctggatatcccgtttattgctttcacggcgaataacgacaattgggttaagcaagacgaggttatcaccctgctgtctaacattcgttccaatcgctgtaaaatctatagcttgctgggcagcagccacgacttgagcgaaaatctggtcgtgctgcgcaacttctaccagagcgtgaccaaagcagcgattgcaatggataacgaccacctggacattgacgtggatatcaccgaaccgagcttcgaacatctgaccatcgcgaccgttaacgaacgtcgtatgcgtattgagattgagaatcaggccatttccctgagctaa

SEQ ID NO: 15 - DNA, 3588 bp, Artificial Sequence
  Codon-optimised operon
catgggaaggagatatagatatgaacaagaaaatcagcttcatcatcaacggtcgcgtagaaatttttccggagtctgatgacctggttcaaagcatcaattttggtgacaatagcgtccacctgccggtgctgaacgatagccaagtgaaaaacattatcgactataatgagaataatgagctgcaactgcacaatatcattaactttctgtataccgtcggtcagcgctggaaaaacgaagaatacagccgtcgtcgtacctatattcgcgatctgaagcgttatatgggctacagcgaggaaatggcgaaactggaagccaattggattagcatgattctgtgctctaaaggtggtttgtacgatctggtgaaaaatgagctgggcagccgtcacattatggacgaatggctgccgcaagacgaaagctacatccgtgccttcccgaaaggcaagagcgttcatctgctgaccggtaatgtcccgctgtcgggcgtgctgtccatcctgcgcgcgattctgaccaagaaccagtgcatcattaagacgagcagcacggatcctttcacggcgaatgcgctggcgctgagcttcatcgacgttgacccacatcacccggtgacccgtagcctgtctgtcgtttattggcagcaccaaggtgacatcagcttggcgaaagagattatgcagcacgccgatgtggtcgttgcctggggtggtgaggatgcaattaactgggcggttaaacacgcaccgccggatatcgacgtcatgaaattcggtccgaaaaagagcttctgcatcattgacaacccggttgacttggttagcgcagcgaccggcgcagcacacgacgtctgtttttacgatcagcaggcatgctttagcacgcagaacatctactacatgggctcccattacgaggagtttaagctggctttgatcgaaaaactgaatctgtatgcacatatcctgcctaacaccaagaaggatttcgacgaaaaggcagcttattccttggtgcaaaaggagtgtctgttcgccggtttgaaagtggaagttgacgttcatcaacgctggatggttattgaatccaatgctggcgttgagctgaaccagccgctgggtcgttgtgtgtacttgcatcacgtggataacatcgagcagattttgccgtatgtgcgtaagaacaaaacccagacgattagcgtgtttccgtgggaggctgcgctgaagtaccgcgatctgctggccctgaaaggcgcggagcgtattgttgaggcgggtatgaataacattttccgtgtgggtggtgcgcacgatggcatgcgtccgctgcaacgcctggtcacttacattagccacgagcgtccgagccattacaccgcgaaggacgtcgcggtcgaaatcgaacagacgcgctttctggaagaggacaagttcctggtgtttgttccataagaattctaacactgtataacattaagaaggaggtaaaagatatgactagctacgtcgacaaacaggaaatcaccgcgagcagcgagattgacgacctgatcttttccagcgatccgttggtgtggtcctatgatgagcaagaaaagattcgcaagaaactggtcctggatgcgttccgccaccactacaagcactgtcaagagtaccgtcattattgccaagcccataaagtcgacgataacattacggaaattgacgatatcccggttttcccgacctctgttttcaagttcacccgtctgctgacctccaacgagaatgagattgagagctggtttacttcgagcggtaccaatggtctgaaaagccaagtcccgcgtgatcgtctgagcattgaacgtctgctgggcagcgtgagctacggcatgaagtacatcggttcgtggtttgaccatcaaatggagctggttaacttgggtccggatcgctttaatgcccacaacatttggttcaagtacgttatgagcctggttgagctgttgtatccgacgagcttcaccgtgacggaagagcacatcgacttcgtgcagacgctgaacagcctggaacgcattaaacatcagggcaaagacatttgtctgatcggttctccgtatttcatctatctgctgtgccgttacatgaaggacaagaacatcagctttagcggtgacaagagcctgtatatcatcaccggtggcggttggaaaagctacgaaaaagagtccctgaagcgtaatgactttaatcacctgttgttcgatacgttcaatctgagcaacattaaccagatccgtgacatctttaaccaggtcgaactgaatacctgtttctttgaggacgagatgcagcgcaaacacgtcccgccgtgggtatacgcgcgtgcgctggatcctgaaaccttgaaaccggttccagatggcatgcctggtctgatgagctatatggatgctagctctacgagctacccggcatttatcgtgaccgacgatattggtattatcagccgcgagtacggtcaatatccgggcgtgctggttgaaattctgcgtcgtgtgaatacccgcaagcagaaaggctgcgcgttgtctctgacggaggcattcggttcctaaaagctttaacactgtataacattaagaaggaggtaaatataatggaaaacgagagcaagtacaaaacgatcgaccacgtaatctgcgtggagggtaacaaaaagattcacgtgtgggagactttgccagaagagaacagcccgaaacgcaaaaacgcaatcattatcgcgagcggtttcgcacgccgcatggatcattttgcgggcctggccgaatacctgagccgtaacggcttccacgttatccgttatgacagcctgcatcacgtcggcctgtcgtctggtaccatcgacgagttcacgatgagcatcggcaagcaaagcctgttggcggttgttgattggctgaccacgcgtaagatcaacaattttggtatgctggcttccagcctgtccgcacgcattgcgtacgcttctctgagcgagattaatgccagctttctgatcaccgccgtgggtttcgtcaatctgcgttatagcctggagcgtgcgctgggtttcgattacttgagcctgccgattaacgagctgccgaataatctggactttgaaggccataagttgggtgcggaggtctttgcgcgtgattgcctggattttggttgggaagatctggcatcgacgattaacaatatgatgtatctggatatcccgtttattgctttcacggcgaataacgacaattgggttaagcaagacgaggttatcaccctgctgtctaacattcgttccaatcgctgtaaaatctatagcttgctgggcagcagccacgacttgagcgaaaatctggtcgtgctgcgcaacttctaccagagcgtgaccaaagcagcgattgcaatggataacgaccacctggacattgacgtggatatcaccgaaccgagcttcgaacatctgaccatcgcgaccgttaacgaacgtcgtatgcgtattgagattgagaatcaggccatttccctgagctaagcggcc

SEQ ID NO: 16 - DNA, 7511 bp, Artificial Sequence
  Expression vector
aacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaattaatacgactcactataggggaattgtgagcggataacaattcccctgtagaaataattttgtttaactttaataaggagatataccatgggaaggagatatagatatgaacaagaaaatcagcttcatcatcaacggtcgcgtagaaatttttccggagtctgatgacctggttcaaagcatcaattttggtgacaatagcgtccacctgccggtgctgaacgatagccaagtgaaaaacattatcgactataatgagaataatgagctgcaactgcacaatatcattaactttctgtataccgtcggtcagcgctggaaaaacgaagaatacagccgtcgtcgtacctatattcgcgatctgaagcgttatatgggctacagcgaggaaatggcgaaactggaagccaattggattagcatgattctgtgctctaaaggtggtttgtacgatctggtgaaaaatgagctgggcagccgtcacattatggacgaatggctgccgcaagacgaaagctacatccgtgccttcccgaaaggcaagagcgttcatctgctgaccggtaatgtcccgctgtcgggcgtgctgtccatcctgcgcgcgattctgaccaagaaccagtgcatcattaagacgagcagcacggatcctttcacggcgaatgcgctggcgctgagcttcatcgacgttgacccacatcacccggtgacccgtagcctgtctgtcgtttattggcagcaccaaggtgacatcagcttggcgaaagagattatgcagcacgccgatgtggtcgttgcctggggtggtgaggatgcaattaactgggcggttaaacacgcaccgccggatatcgacgtcatgaaattcggtccgaaaaagagcttctgcatcattgacaacccggttgacttggttagcgcagcgaccggcgcagcacacgacgtctgtttttacgatcagcaggcatgctttagcacgcagaacatctactacatgggctcccattacgaggagtttaagctggctttgatcgaaaaactgaatctgtatgcacatatcctgcctaacaccaagaaggatttcgacgaaaaggcagcttattccttggtgcaaaaggagtgtctgttcgccggtttgaaagtggaagttgacgttcatcaacgctggatggttattgaatccaatgctggcgttgagctgaaccagccgctgggtcgttgtgtgtacttgcatcacgtggataacatcgagcagattttgccgtatgtgcgtaagaacaaaacccagacgattagcgtgtttccgtgggaggctgcgctgaagtaccgcgatctgctggccctgaaaggcgcggagcgtattgttgaggcgggtatgaataacattttccgtgtgggtggtgcgcacgatggcatgcgtccgctgcaacgcctggtcacttacattagccacgagcgtccgagccattacaccgcgaaggacgtcgcggtcgaaatcgaacagacgcgctttctggaagaggacaagttcctggtgtttgttccataagaattctaacactgtataacattaagaaggaggtaaaagatatgactagctacgtcgacaaacaggaaatcaccgcgagcagcgagattgacgacctgatcttttccagcgatccgttggtgtggtcctatgatgagcaagaaaagattcgcaagaaactggtcctggatgcgttccgccaccactacaagcactgtcaagagtaccgtcattattgccaagcccataaagtcgacgataacattacggaaattgacgatatcccggttttcccgacctctgttttcaagttcacccgtctgctgacctccaacgagaatgagattgagagctggtttacttcgagcggtaccaatggtctgaaaagccaagtcccgcgtgatcgtctgagcattgaacgtctgctgggcagcgtgagctacggcatgaagtacatcggttcgtggtttgaccatcaaatggagctggttaacttgggtccggatcgctttaatgcccacaacatttggttcaagtacgttatgagcctggttgagctgttgtatccgacgagcttcaccgtgacggaagagcacatcgacttcgtgcagacgctgaacagcctggaacgcattaaacatcagggcaaagacatttgtctgatcggttctccgtatttcatctatctgctgtgccgttacatgaaggacaagaacatcagctttagcggtgacaagagcctgtatatcatcaccggtggcggttggaaaagctacgaaaaagagtccctgaagcgtaatgactttaatcacctgttgttcgatacgttcaatctgagcaacattaaccagatccgtgacatctttaaccaggtcgaactgaatacctgtttctttgaggacgagatgcagcgcaaacacgtcccgccgtgggtatacgcgcgtgcgctggatcctgaaaccttgaaaccggttccagatggcatgcctggtctgatgagctatatggatgctagctctacgagctacccggcatttatcgtgaccgacgatattggtattatcagccgcgagtacggtcaatatccgggcgtgctggttgaaattctgcgtcgtgtgaatacccgcaagcagaaaggctgcgcgttgtctctgacggaggcattcggttcctaaaagctttaacactgtataacattaagaaggaggtaaatataatggaaaacgagagcaagtacaaaacgatcgaccacgtaatctgcgtggagggtaacaaaaagattcacgtgtgggagactttgccagaagagaacagcccgaaacgcaaaaacgcaatcattatcgcgagcggtttcgcacgccgcatggatcattttgcgggcctggccgaatacctgagccgtaacggcttccacgttatccgttatgacagcctgcatcacgtcggcctgtcgtctggtaccatcgacgagttcacgatgagcatcggcaagcaaagcctgttggcggttgttgattggctgaccacgcgtaagatcaacaattttggtatgctggcttccagcctgtccgcacgcattgcgtacgcttctctgagcgagattaatgccagctttctgatcaccgccgtgggtttcgtcaatctgcgttatagcctggagcgtgcgctgggtttcgattacttgagcctgccgattaacgagctgccgaataatctggactttgaaggccataagttgggtgcggaggtctttgcgcgtgattgcctggattttggttgggaagatctggcatcgacgattaacaatatgatgtatctggatatcccgtttattgctttcacggcgaataacgacaattgggttaagcaagacgaggttatcaccctgctgtctaacattcgttccaatcgctgtaaaatctatagcttgctgggcagcagccacgacttgagcgaaaatctggtcgtgctgcgcaacttctaccagagcgtgaccaaagcagcgattgcaatggataacgaccacctggacattgacgtggatatcaccgaaccgagcttcgaacatctgaccatcgcgaccgttaacgaacgtcgtatgcgtattgagattgagaatcaggccatttccctgagctaagcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatggcagatctcaattggatatcggccggccacgcgatcgctgacgtcggtaccctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatagtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccgg

SEQ ID NO: 17 - DNA, 906 bp, Cinnamomum camphora
atgggtctggaatggaaaccgaagccgaatccgccacaactgctggatgatcatttcggtccgcacggcctggtctttcgccgtaccttcgcaatccgtagctatgaggttggcccggaccgcagcacgtctatcgtggctgttatgaatcacctgcaagaggccgctttgaaccatgcgaaaagcgtcggcattctgggcgatggcttcggtaccactttggaaatgagcaagcgcgatctgatctgggtggttaaacgtacgcacgttgccgtggaacgttacccggcgtggggtgataccgtagaagttgagtgctgggtcggcgcaagcggtaataacggtcgccgtcacgactttctggtgcgtgactgtaaaaccggtgaaatcttgacgcgctgtaccagcctgagcgttatgatgaacacccgcacgcgtcgtctgtccaagattcctgaagaggtccgtggtgagattggtccggcgttcatcgacaacgtggccgttaaggacgaagagattaagaagccgcagaaattgaatgactctacggcggattacattcagggtggtctgacgccgcgttggaatgacctggacattaaccagcacgtgaacaatatcaaatatgtcgattggattctggaaaccgtgccggacagcatttttgagtcgcatcacatcagcagcttcaccattgagtaccgtcgcgagtgcacgatggatagcgttctgcaaagcctgaccactgtgagcggcggtagctctgaggcgggtctggtgtgcgagcatctgctgcagctggagggtggcagcgaagttctgcgtgcaaaaaccgagtggcgtccgaagctgaccgactcctttcgtggcatctccgtcatcccagcggaaagcagcgtctaa

SEQ ID NO: 18 - DNA, 6291 bp, Artificial Sequence
  Expression vector
ggggaattgtgagcggataacaattcccctctagaaataattttgtttaactttaagaaggagatataccatgggtctggaatggaaaccgaagccgaatccgccacaactgctggatgatcatttcggtccgcacggcctggtctttcgccgtaccttcgcaatccgtagctatgaggttggcccggaccgcagcacgtctatcgtggctgttatgaatcacctgcaagaggccgctttgaaccatgcgaaaagcgtcggcattctgggcgatggcttcggtaccactttggaaatgagcaagcgcgatctgatctgggtggttaaacgtacgcacgttgccgtggaacgttacccggcgtggggtgataccgtagaagttgagtgctgggtcggcgcaagcggtaataacggtcgccgtcacgactttctggtgcgtgactgtaaaaccggtgaaatcttgacgcgctgtaccagcctgagcgttatgatgaacacccgcacgcgtcgtctgtccaagattcctgaagaggtccgtggtgagattggtccggcgttcatcgacaacgtggccgttaaggacgaagagattaagaagccgcagaaattgaatgactctacggcggattacattcagggtggtctgacgccgcgttggaatgacctggacattaaccagcacgtgaacaatatcaaatatgtcgattggattctggaaaccgtgccggacagcatttttgagtcgcatcacatcagcagcttcaccattgagtaccgtcgcgagtgcacgatggatagcgttctgcaaagcctgaccactgtgagcggcggtagctctgaggcgggtctggtgtgcgagcatctgctgcagctggagggtggcagcgaagttctgcgtgcaaaaaccgagtggcgtccgaagctgaccgactcctttcgtggcatctccgtcatcccagcggaaagcagcgtctaaggatccgaattcgagctcggcgcgcctgcaggtcgacaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatggcagatctcaattggatatcggccggccacgcgatcgctgacgtcggtaccctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactata

SEQ ID NO: 19 - DNA, 978 bp, Bacillus subtilis
atgagcaaggcgaaaatcacggcaatcggcacctacgcaccaagccgtcgtctgaccaatgcggatctggagaagattgttgacacctctgatgaatggatcgttcaacgtacgggtatgcgtgaacgtcgtattgccgacgaacatcagttcacgtctgatctgtgcatcgaagccgttaagaacctgaaaagccgttacaaaggcacgctggatgacgttgacatgatcctggttgcaaccacgacctctgactatgcttttccgagcaccgcttgtcgtgtgcaggagtatttcggctgggaatccactggtgcgctggatatcaatgccacctgtgcgggtctgacctacggtctgcacctggccaatggcctgattaccagcggcctgcatcaaaagattctggttattgcgggcgaaacgctgagcaaagttaccgattacaccgatcgcacgacctgcgttttgtttggcgacgcagcgggtgcactgctggttgagcgcgatgaggaaacgccaggtttcctggcgagcgtccagggcactagcggtaacggtggtgacatcctgtaccgtgcaggtctgcgtaacgagattaacggtgtgcagctggtgggctctggcaagatggtgcaaaatggccgtgaggtttacaagtgggctgcgcgcactgttccgggcgagttcgagcgcctgctgcacaaagcaggtctgagcagcgacgatctggactggtttgtgccgcacagcgccaacctgcgtatgatcgagagcatctgcgaaaagacgccgttcccaatcgaaaagaccttgacgagcgtggagcattacggtaataccagctccgtgtctattgtcctggcgctggacttggcagtgaaggcaggcaaactgaaaaaggatcagatcgttctgctgtttggcttcggtggtggcttgacctacacgggcctgctgatcaaatggggtatgtaa

SEQ ID NO: 20 - DNA, 993 bp, Bacillus subtilis
atgggcacgaaccgccaccaagcactgggcctgaccgaccaagaggcggttgatatgtaccgcacgatgctgctggcgcgcaagattgatgagcgtatgtggctgttgaatcgttccggcaagattccatttgtgatttcttgccagggccaagaggcagcacaagttggtgcagcgttcgcgctggatcgtgagatggattacgtgctgccgtactaccgtgatatgggtgtggtgctggcattcggtatgaccgcaaaagatctgatgatgtctggctttgcaaaagcggcggacccaaacagcggcggtcgccagatgccaggtcactttggtcagaagaagaatcgtattgtcaccggtagcagcccggttacgacgcaggttccgcacgcggttggtattgcgctggccggtcgtatggaaaagaaagatatcgccgcgttcgtcacgtttggcgagggtagcagcaatcagggtgactttcatgagggtgccaacttcgctgcggtccataaactgccggtcatcttcatgtgcgaaaacaacaagtacgccattagcgttccgtacgacaagcaggttgcttgcgagaacatcagcgaccgcgcgatcggctatggtatgccgggtgtgacggtcaacggcaacgatccgctggaggtttatcaagcggttaaagaagcgcgcgagcgtgcccgtcgcggtgagggtccgacgttgatcgaaaccatttcctatcgtctgacgcctcacagcagcgatgatgatgacagcagctaccgtggtcgtgaagaggtcgaagaggccaaaaagagcgacccgctgctgacctaccaagcgtatctgaaagaaacgggtctgctgagcgacgagattgagcaaaccatgctggacgagatcatggcaatcgtgaatgaggcaaccgacgaggcggagaacgcgccgtatgcggcaccggaaagcgcactggattatgtctacgcgaagtaa

SEQ ID NO: 21 - DNA, 984 bp, Bacillus subtilis
atgagcgtaatgagctacatcgatgcaatcaacctggccatgaaagaagaaatggaacgcgacagccgcgtttttgttttgggtgaggacgtcggtcgcaaaggtggtgtgttcaaagccaccgcgggtttgtacgagcaatttggcgaagagcgtgtcatggatacgccgctggccgaaagcgctattgcaggcgtcggcatcggtgcggctatgtatggtatgcgtccgatcgctgaaatgcaatttgcagactttatcatgccagccgtcaaccagatcatcagcgaggcagcgaaaatccgttatcgtagcaacaacgattggagctgtccgatcgttgtccgtgccccgtatggtggtggtgttcacggcgcactgtatcatagccagagcgttgaagcgattttcgcaaaccaacctggtctgaaaatcgttatgccaagcaccccgtacgatgcgaagggtttgctgaaagcggcggtgcgcgatgaagatccggtgctgttcttcgagcacaagcgtgcgtaccgtctgattaaaggcgaggtcccggcagacgactacgtcttgccgatcggtaaagcggatgttaagcgtgaaggtgatgatatcaccgtgatcacgtacggcctgtgcgtgcacttcgccctgcaagcggccgaacgcctggagaaggacggcatcagcgcacacgttgtagacctgcgtaccgtctacccgttggataaagaagccatcatcgaggcggcgagcaaaaccggcaaggtgctgctggtcacggaagataccaaagaaggtagcatcatgagcgaggttgcagccatcattagcgagcactgtttgttcgacttggatgcgccgattaagcgtctggcgggtccagatatcccggccatgccgtacgcaccgacgatggagaaatactttatggtcaacccggataaggtggaagcggccatgcgtgagctggcggagttctaa

SEQ ID NO: 22 - DNA, 1275 bp, Bacillus subtilis
atggccatcgagcaaatgaccatgccgcaactgggcgagagcgtaacggaaggcaccatttccaaatggctggttgctccaggtgataaagtcaacaagtatgacccgatcgctgaggttatgaccgataaggtgaacgcggaggttccgtcctctttcactggcaccattaccgaactggtcggcgaagagggtcaaacgctgcaagtcggcgagatgatctgtaagattgaaacggagggtgctaatccggctgaacaaaagcaggagcaaccggcagcgtctgaagcggcagaaaatccagtcgcgaagagcgcgggtgccgcagatcaaccgaacaaaaagcgttacagcccggcagttttgcgcctggctggtgagcacggcatcgacctggatcaagtgactggtacgggcgcaggtggccgcattacccgtaaggacatccaacgcttgattgaaacgggtggtgtccaggaacagaacccggaggagctgaaaaccgccgcaccggcaccgaaaagcgcgagcaaaccggagccgaaggaagaaacctcttacccggcgtccgctgcgggcgataaggagattccggttactggcgttcgcaaggccatcgctagcaatatgaagcgcagcaagactgagatcccgcacgcatggacgatgatggaggtggatgtgaccaacatggtagcataccgtaatagcatcaaggatagcttcaaaaagaccgaaggtttcaacctgacgttctttgccttctttgtgaaggccgttgcacaggcactgaaagagtttccgcaaatgaacagcatgtgggctggcgacaagattattcaaaagaaggatatcaacattagcattgcagtcgccaccgaggacagcctgttcgtgccggtaatcaaaaatgctgatgaaaagactatcaaaggtattgcaaaggacatcaccggcctggcgaagaaagttcgcgacggtaagctgaccgcagatgacatgcagggtggcacctttacggtcaacaacacgggcagctttggcagcgtccagagcatgggtattatcaactatccgcaggcggcaattctgcaagttgaatccatcgtgaaacgcccggttgttatggacaacggcatgattgcagttcgtgacatggtaaacttgtgtctgagcttggaccaccgcgttctggacggcctggtctgcggtcgtttcttgggccgtgtgaaacagatcctggagagcattgatgagaaaacgagcgtgtattaa

SEQ ID NO: 23 - DNA, 1425 bp, Bacillus subtilis
atggcaacggagtacgacgtagtgattttgggcggtggcacgggcggttacgtggcggccattcgtgcggcgcaattgggcctgaaaacggccgtggtcgaaaaagaaaaactgggcggcacctgcctgcacaagggttgtattccgagcaaagccctgttgcgttccgcggaggtgtaccgtaccgctcgtgaagcggaccaattcggcgtggaaaccgcgggtgtgtccctgaactttgagaaagtccagcagcgtaaacaggcggtggtggacaaactggctgcgggtgtcaatcacctgatgaagaagggtaaaatcgatgtgtataccggttatggccgcatcctgggtccgagcattttcagcccgctgccgggtactatttccgtggaacgtggcaacggtgaagaaaacgacatgttgatccctaaacaggtgatcatcgcgaccggtagccgtccgcgcatgctgccaggtctggaagttgacggtaaaagcgtgctgaccagcgatgaggcgctgcaaatggaggagttgccgcagagcatcatcattgtaggtggcggcgtcattggcattgagtgggcgagcatgctgcatgattttggcgtcaaagtcactgtgatcgagtacgccgaccgtattctgccgacggaggatttggagatttccaaagaaatggaaagcctgctgaaaaagaaaggtatccaattcattaccggtgctaaggttctgccggacacgatgaccaaaactagcgacgatatcagcattcaagcagaaaaagatggcgaaacggtcacctacagcgcggagaaaatgttggtgagcatcggtcgtcaggcgaatatcgagggtattggtctggaaaacaccgacattgttaccgagaatggtatgatctccgtcaacgagagctgccaaacgaaagagtcgcacatctatgccatcggtgacgtcatcggtggcctgcaattggcccacgtcgcaagccatgagggtatcatcgcagtagaacatttcgccggtctgaatccgcacccgctggacccgactctggtccctaagtgtatctactccagcccggaagccgctagcgtaggtctgaccgaagatgaggctaaggcgaatggccacaacgtcaagattggcaagttcccgtttatggctattggtaaggcgctggtgtatggcgagagcgacggttttgtcaagattgtagctgatcgtgataccgacgatattctgggtgtgcacatgatcggtccgcacgtgaccgacatgattagcgaagcaggtctggccaaagtactggacgcgaccccgtgggaagtaggccagaccattcacccgcatcctacgctgagcgaagcgattggtgaggcggcattggccgcagacggtaaagctatccacttctaa

SEQ ID NO: 24 - DNA, 5862 bp, Artificial Sequence
  Codon-optimised operon
ccatgggaaggagatataccatgggcacgaaccgccaccaagcactgggcctgaccgaccaagaggcggttgatatgtaccgcacgatgctgctggcgcgcaagattgatgagcgtatgtggctgttgaatcgttccggcaagattccatttgtgatttcttgccagggccaagaggcagcacaagttggtgcagcgttcgcgctggatcgtgagatggattacgtgctgccgtactaccgtgatatgggtgtggtgctggcattcggtatgaccgcaaaagatctgatgatgtctggctttgcaaaagcggcggacccaaacagcggcggtcgccagatgccaggtcactttggtcagaagaagaatcgtattgtcaccggtagcagcccggttacgacgcaggttccgcacgcggttggtattgcgctggccggtcgtatggaaaagaaagatatcgccgcgttcgtcacgtttggcgagggtagcagcaatcagggtgactttcatgagggtgccaacttcgctgcggtccataaactgccggtcatcttcatgtgcgaaaacaacaagtacgccattagcgttccgtacgacaagcaggttgcttgcgagaacatcagcgaccgcgcgatcggctatggtatgccgggtgtgacggtcaacggcaacgatccgctggaggtttatcaagcggttaaagaagcgcgcgagcgtgcccgtcgcggtgagggtccgacgttgatcgaaaccatttcctatcgtctgacgcctcacagcagcgatgatgatgacagcagctaccgtggtcgtgaagaggtcgaagaggccaaaaagagcgacccgctgctgacctaccaagcgtatctgaaagaaacgggtctgctgagcgacgagattgagcaaaccatgctggacgagatcatggcaatcgtgaatgaggcaaccgacgaggcggagaacgcgccgtatgcggcaccggaaagcgcactggattatgtctacgcgaagtaaggatcccactgtataacattaagaaggaggtaaaaaaaatgagcgtaatgagctacatcgatgcaatcaacctggccatgaaagaagaaatggaacgcgacagccgcgtttttgttttgggtgaggacgtcggtcgcaaaggtggtgtgttcaaagccaccgcgggtttgtacgagcaatttggcgaagagcgtgtcatggatacgccgctggccgaaagcgctattgcaggcgtcggcatcggtgcggctatgtatggtatgcgtccgatcgctgaaatgcaatttgcagactttatcatgccagccgtcaaccagatcatcagcgaggcagcgaaaatccgttatcgtagcaacaacgattggagctgtccgatcgttgtccgtgccccgtatggtggtggtgttcacggcgcactgtatcatagccagagcgttgaagcgattttcgcaaaccaacctggtctgaaaatcgttatgccaagcaccccgtacgatgcgaagggtttgctgaaagcggcggtgcgcgatgaagatccggtgctgttcttcgagcacaagcgtgcgtaccgtctgattaaaggcgaggtcccggcagacgactacgtcttgccgatcggtaaagcggatgttaagcgtgaaggtgatgatatcaccgtgatcacgtacggcctgtgcgtgcacttcgccctgcaagcggccgaacgcctggagaaggacggcatcagcgcacacgttgtagacctgcgtaccgtctacccgttggataaagaagccatcatcgaggcggcgagcaaaaccggcaaggtgctgctggtcacggaagataccaaagaaggtagcatcatgagcgaggttgcagccatcattagcgagcactgtttgttcgacttggatgcgccgattaagcgtctggcgggtccagatatcccggccatgccgtacgcaccgacgatggagaaatactttatggtcaacccggataaggtggaagcggccatgcgtgagctggcggagttctaaggatccgaattcactgtataacattaagaaggaggtaaaaaaaatggccatcgagcaaatgaccatgccgcaactgggcgagagcgtaacggaaggcaccatttccaaatggctggttgctccaggtgataaagtcaacaagtatgacccgatcgctgaggttatgaccgataaggtgaacgcggaggttccgtcctctttcactggcaccattaccgaactggtcggcgaagagggtcaaacgctgcaagtcggcgagatgatctgtaagattgaaacggagggtgctaatccggctgaacaaaagcaggagcaaccggcagcgtctgaagcggcagaaaatccagtcgcgaagagcgcgggtgccgcagatcaaccgaacaaaaagcgttacagcccggcagttttgcgcctggctggtgagcacggcatcgacctggatcaagtgactggtacgggcgcaggtggccgcattacccgtaaggacatccaacgcttgattgaaacgggtggtgtccaggaacagaacccggaggagctgaaaaccgccgcaccggcaccgaaaagcgcgagcaaaccggagccgaaggaagaaacctcttacccggcgtccgctgcgggcgataaggagattccggttactggcgttcgcaaggccatcgctagcaatatgaagcgcagcaagactgagatcccgcacgcatggacgatgatggaggtggatgtgaccaacatggtagcataccgtaatagcatcaaggatagcttcaaaaagaccgaaggtttcaacctgacgttctttgccttctttgtgaaggccgttgcacaggcactgaaagagtttccgcaaatgaacagcatgtgggctggcgacaagattattcaaaagaaggatatcaacattagcattgcagtcgccaccgaggacagcctgttcgtgccggtaatcaaaaatgctgatgaaaagactatcaaaggtattgcaaaggacatcaccggcctggcgaagaaagttcgcgacggtaagctgaccgcagatgacatgcagggtggcacctttacggtcaacaacacgggcagctttggcagcgtccagagcatgggtattatcaactatccgcaggcggcaattctgcaagttgaatccatcgtgaaacgcccggttgttatggacaacggcatgattgcagttcgtgacatggtaaacttgtgtctgagcttggaccaccgcgttctggacggcctggtctgcggtcgtttcttgggccgtgtgaaacagatcctggagagcattgatgagaaaacgagcgtgtattaagaattcgagctcactgtataacattaagaaggaggtaaaaaaaatggcaacggagtacgacgtagtgattttgggcggtggcacgggcggttacgtggcggccattcgtgcggcgcaattgggcctgaaaacggccgtggtcgaaaaagaaaaactgggcggcacctgcctgcacaagggttgtattccgagcaaagccctgttgcgttccgcggaggtgtaccgtaccgctcgtgaagcggaccaattcggcgtggaaaccgcgggtgtgtccctgaactttgagaaagtccagcagcgtaaacaggcggtggtggacaaactggctgcgggtgtcaatcacctgatgaagaagggtaaaatcgatgtgtataccggttatggccgcatcctgggtccgagcattttcagcccgctgccgggtactatttccgtggaacgtggcaacggtgaagaaaacgacatgttgatccctaaacaggtgatcatcgcgaccggtagccgtccgcgcatgctgccaggtctggaagttgacggtaaaagcgtgctgaccagcgatgaggcgctgcaaatggaggagttgccgcagagcatcatcattgtaggtggcggcgtcattggcattgagtgggcgagcatgctgcatgattttggcgtcaaagtcactgtgatcgagtacgccgaccgtattctgccgacggaggatttggagatttccaaagaaatggaaagcctgctgaaaaagaaaggtatccaattcattaccggtgctaaggttctgccggacacgatgaccaaaactagcgacgatatcagcattcaagcagaaaaagatggcgaaacggtcacctacagcgcggagaaaatgttggtgagcatcggtcgtcaggcgaatatcgagggtattggtctggaaaacaccgacattgttaccgagaatggtatgatctccgtcaacgagagctgccaaacgaaagagtcgcacatctatgccatcggtgacgtcatcggtggcctgcaattggcccacgtcgcaagccatgagggtatcatcgcagtagaacatttcgccggtctgaatccgcacccgctggacccgactctggtccctaagtgtatctactccagcccggaagccgctagcgtaggtctgaccgaagatgaggctaaggcgaatggccacaacgtcaagattggcaagttcccgtttatggctattggtaaggcgctggtgtatggcgagagcgacggttttgtcaagattgtagctgatcgtgataccgacgatattctgggtgtgcacatgatcggtccgcacgtgaccgacatgattagcgaagcaggtctggccaaagtactggacgcgaccccgtgggaagtaggccagaccattcacccgcatcctacgctgagcgaagcgattggtgaggcggcattggccgcagacggtaaagctatccacttctaagagctcgtcgaccactgtataacattaagaaggaggtaaaaaaaatgagcaaggcgaaaatcacggcaatcggcacctacgcaccaagccgtcgtctgaccaatgcggatctggagaagattgttgacacctctgatgaatggatcgttcaacgtacgggtatgcgtgaacgtcgtattgccgacgaacatcagttcacgtctgatctgtgcatcgaagccgttaagaacctgaaaagccgttacaaaggcacgctggatgacgttgacatgatcctggttgcaaccacgacctctgactatgcttttccgagcaccgcttgtcgtgtgcaggagtatttcggctgggaatccactggtgcgctggatatcaatgccacctgtgcgggtctgacctacggtctgcacctggccaatggcctgattaccagcggcctgcatcaaaagattctggttattgcgggcgaaacgctgagcaaagttaccgattacaccgatcgcacgacctgcgttttgtttggcgacgcagcgggtgcactgctggttgagcgcgatgaggaaacgccaggtttcctggcgagcgtccagggcactagcggtaacggtggtgacatcctgtaccgtgcaggtctgcgtaacgagattaacggtgtgcagctggtgggctctggcaagatggtgcaaaatggccgtgaggtttacaagtgggctgcgcgcactgttccgggcgagttcgagcgcctgctgcacaaagcaggtctgagcagcgacgatctggactggtttgtgccgcacagcgccaacctgcgtatgatcgagagcatctgcgaaaagacgccgttcccaatcgaaaagaccttgacgagcgtggagcattacggtaataccagctccgtgtctattgtcctggcgctggacttggcagtgaaggcaggcaaactgaaaaaggatcagatcgttctgctgtttggcttcggtggtggcttgacctacacgggcctgctgatcaaatggggtatgtaatgagtcgacgcggccgcgc

SEQ ID NO: 25 - DNA, 11200 bp, Artificial Sequence
  Expression vector
ggggaattgtgagcggataacaattcccctctagaaataattttgtttaactttaagaaggagatataccatgggaaggagatataccatgggcacgaaccgccaccaagcactgggcctgaccgaccaagaggcggttgatatgtaccgcacgatgctgctggcgcgcaagattgatgagcgtatgtggctgttgaatcgttccggcaagattccatttgtgatttcttgccagggccaagaggcagcacaagttggtgcagcgttcgcgctggatcgtgagatggattacgtgctgccgtactaccgtgatatgggtgtggtgctggcattcggtatgaccgcaaaagatctgatgatgtctggctttgcaaaagcggcggacccaaacagcggcggtcgccagatgccaggtcactttggtcagaagaagaatcgtattgtcaccggtagcagcccggttacgacgcaggttccgcacgcggttggtattgcgctggccggtcgtatggaaaagaaagatatcgccgcgttcgtcacgtttggcgagggtagcagcaatcagggtgactttcatgagggtgccaacttcgctgcggtccataaactgccggtcatcttcatgtgcgaaaacaacaagtacgccattagcgttccgtacgacaagcaggttgcttgcgagaacatcagcgaccgcgcgatcggctatggtatgccgggtgtgacggtcaacggcaacgatccgctggaggtttatcaagcggttaaagaagcgcgcgagcgtgcccgtcgcggtgagggtccgacgttgatcgaaaccatttcctatcgtctgacgcctcacagcagcgatgatgatgacagcagctaccgtggtcgtgaagaggtcgaagaggccaaaaagagcgacccgctgctgacctaccaagcgtatctgaaagaaacgggtctgctgagcgacgagattgagcaaaccatgctggacgagatcatggcaatcgtgaatgaggcaaccgacgaggcggagaacgcgccgtatgcggcaccggaaagcgcactggattatgtctacgcgaagtaaggatcccactgtataacattaagaaggaggtaaaaaaaatgagcgtaatgagctacatcgatgcaatcaacctggccatgaaagaagaaatggaacgcgacagccgcgtttttgttttgggtgaggacgtcggtcgcaaaggtggtgtgttcaaagccaccgcgggtttgtacgagcaatttggcgaagagcgtgtcatggatacgccgctggccgaaagcgctattgcaggcgtcggcatcggtgcggctatgtatggtatgcgtccgatcgctgaaatgcaatttgcagactttatcatgccagccgtcaaccagatcatcagcgaggcagcgaaaatccgttatcgtagcaacaacgattggagctgtccgatcgttgtccgtgccccgtatggtggtggtgttcacggcgcactgtatcatagccagagcgttgaagcgattttcgcaaaccaacctggtctgaaaatcgttatgccaagcaccccgtacgatgcgaagggtttgctgaaagcggcggtgcgcgatgaagatccggtgctgttcttcgagcacaagcgtgcgtaccgtctgattaaaggcgaggtcccggcagacgactacgtcttgccgatcggtaaagcggatgttaagcgtgaaggtgatgatatcaccgtgatcacgtacggcctgtgcgtgcacttcgccctgcaagcggccgaacgcctggagaaggacggcatcagcgcacacgttgtagacctgcgtaccgtctacccgttggataaagaagccatcatcgaggcggcgagcaaaaccggcaaggtgctgctggtcacggaagataccaaagaaggtagcatcatgagcgaggttgcagccatcattagcgagcactgtttgttcgacttggatgcgccgattaagcgtctggcgggtccagatatcccggccatgccgtacgcaccgacgatggagaaatactttatggtcaacccggataaggtggaagcggccatgcgtgagctggcggagttctaaggatccgaattcactgtataacattaagaaggaggtaaaaaaaatggccatcgagcaaatgaccatgccgcaactgggcgagagcgtaacggaaggcaccatttccaaatggctggttgctccaggtgataaagtcaacaagtatgacccgatcgctgaggttatgaccgataaggtgaacgcggaggttccgtcctctttcactggcaccattaccgaactggtcggcgaagagggtcaaacgctgcaagtcggcgagatgatctgtaagattgaaacggagggtgctaatccggctgaacaaaagcaggagcaaccggcagcgtctgaagcggcagaaaatccagtcgcgaagagcgcgggtgccgcagatcaaccgaacaaaaagcgttacagcccggcagttttgcgcctggctggtgagcacggcatcgacctggatcaagtgactggtacgggcgcaggtggccgcattacccgtaaggacatccaacgcttgattgaaacgggtggtgtccaggaacagaacccggaggagctgaaaaccgccgcaccggcaccgaaaagcgcgagcaaaccggagccgaaggaagaaacctcttacccggcgtccgctgcgggcgataaggagattccggttactggcgttcgcaaggccatcgctagcaatatgaagcgcagcaagactgagatcccgcacgcatggacgatgatggaggtggatgtgaccaacatggtagcataccgtaatagcatcaaggatagcttcaaaaagaccgaaggtttcaacctgacgttctttgccttctttgtgaaggccgttgcacaggcactgaaagagtttccgcaaatgaacagcatgtgggctggcgacaagattattcaaaagaaggatatcaacattagcattgcagtcgccaccgaggacagcctgttcgtgccggtaatcaaaaatgctgatgaaaagactatcaaaggtattgcaaaggacatcaccggcctggcgaagaaagttcgcgacggtaagctgaccgcagatgacatgcagggtggcacctttacggtcaacaacacgggcagctttggcagcgtccagagcatgggtattatcaactatccgcaggcggcaattctgcaagttgaatccatcgtgaaacgcccggttgttatggacaacggcatgattgcagttcgtgacatggtaaacttgtgtctgagcttggaccaccgcgttctggacggcctggtctgcggtcgtttcttgggccgtgtgaaacagatcctggagagcattgatgagaaaacgagcgtgtattaagaattcgagctcactgtataacattaagaaggaggtaaaaaaaatggcaacggagtacgacgtagtgattttgggcggtggcacgggcggttacgtggcggccattcgtgcggcgcaattgggcctgaaaacggccgtggtcgaaaaagaaaaactgggcggcacctgcctgcacaagggttgtattccgagcaaagccctgttgcgttccgcggaggtgtaccgtaccgctcgtgaagcggaccaattcggcgtggaaaccgcgggtgtgtccctgaactttgagaaagtccagcagcgtaaacaggcggtggtggacaaactggctgcgggtgtcaatcacctgatgaagaagggtaaaatcgatgtgtataccggttatggccgcatcctgggtccgagcattttcagcccgctgccgggtactatttccgtggaacgtggcaacggtgaagaaaacgacatgttgatccctaaacaggtgatcatcgcgaccggtagccgtccgcgcatgctgccaggtctggaagttgacggtaaaagcgtgctgaccagcgatgaggcgctgcaaatggaggagttgccgcagagcatcatcattgtaggtggcggcgtcattggcattgagtgggcgagcatgctgcatgattttggcgtcaaagtcactgtgatcgagtacgccgaccgtattctgccgacggaggatttggagatttccaaagaaatggaaagcctgctgaaaaagaaaggtatccaattcattaccggtgctaaggttctgccggacacgatgaccaaaactagcgacgatatcagcattcaagcagaaaaagatggcgaaacggtcacctacagcgcggagaaaatgttggtgagcatcggtcgtcaggcgaatatcgagggtattggtctggaaaacaccgacattgttaccgagaatggtatgatctccgtcaacgagagctgccaaacgaaagagtcgcacatctatgccatcggtgacgtcatcggtggcctgcaattggcccacgtcgcaagccatgagggtatcatcgcagtagaacatttcgccggtctgaatccgcacccgctggacccgactctggtccctaagtgtatctactccagcccggaagccgctagcgtaggtctgaccgaagatgaggctaaggcgaatggccacaacgtcaagattggcaagttcccgtttatggctattggtaaggcgctggtgtatggcgagagcgacggttttgtcaagattgtagctgatcgtgataccgacgatattctgggtgtgcacatgatcggtccgcacgtgaccgacatgattagcgaagcaggtctggccaaagtactggacgcgaccccgtgggaagtaggccagaccattcacccgcatcctacgctgagcgaagcgattggtgaggcggcattggccgcagacggtaaagctatccacttctaagagctcgtcgaccactgtataacattaagaaggaggtaaaaaaaatgagcaaggcgaaaatcacggcaatcggcacctacgcaccaagccgtcgtctgaccaatgcggatctggagaagattgttgacacctctgatgaatggatcgttcaacgtacgggtatgcgtgaacgtcgtattgccgacgaacatcagttcacgtctgatctgtgcatcgaagccgttaagaacctgaaaagccgttacaaaggcacgctggatgacgttgacatgatcctggttgcaaccacgacctctgactatgcttttccgagcaccgcttgtcgtgtgcaggagtatttcggctgggaatccactggtgcgctggatatcaatgccacctgtgcgggtctgacctacggtctgcacctggccaatggcctgattaccagcggcctgcatcaaaagattctggttattgcgggcgaaacgctgagcaaagttaccgattacaccgatcgcacgacctgcgttttgtttggcgacgcagcgggtgcactgctggttgagcgcgatgaggaaacgccaggtttcctggcgagcgtccagggcactagcggtaacggtggtgacatcctgtaccgtgcaggtctgcgtaacgagattaacggtgtgcagctggtgggctctggcaagatggtgcaaaatggccgtgaggtttacaagtgggctgcgcgcactgttccgggcgagttcgagcgcctgctgcacaaagcaggtctgagcagcgacgatctggactggtttgtgccgcacagcgccaacctgcgtatgatcgagagcatctgcgaaaagacgccgttcccaatcgaaaagaccttgacgagcgtggagcattacggtaataccagctccgtgtctattgtcctggcgctggacttggcagtgaaggcaggcaaactgaaaaaggatcagatcgttctgctgtttggcttcggtggtggcttgacctacacgggcctgctgatcaaatggggtatgtaatgagtcgacgcggccgcgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatggcagatctcaattggatatcggccggccacgcgatcgctgacgtcggtaccctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactata

SEQ ID NO: 26 - DNA, 25 bp, Artificial Sequence
  Primer sequence
catatgcagcagcttacagaccaat

SEQ ID NO: 27 - DNA, 29 bp, Artificial Sequence
  Primer sequence
ctcgagttaagcacctatgagtccgtagg

SEQ ID NO: 28 - protein, 477 aa, Vibrio harveyi
MEKHLPLIVNGQIISTEENRFEISFEEKKVKIDSFNNLHLTQMVNHDYLNDLNINNIINFLYTTGQRWKSEEYSRRRAYIRSLITYLGYSPQMAKLEANWIAMILCSKSALYDIIDTELGSTHIQDEWLPQGECYVRAFPKGRTMHLLAGNVPLSGVTSILRGILTRNQCIVRMSASDPFTAHALAMSFIDVDPNHPISRSISVLYWPHASDTTLAEELLSHMDAVVAWGGRDAIDWAVKHSPSHIDVLKFGPKKSFTVLDHPADLEEAASGVAHDICFYDQNACFSTQNIYFSGDKYEEFKLKLVEKLNLYQEVLPKSKQSFDDEALFSMTRLECQFSGLKVISEPENNWMIIESEPGVEYNHPLSRCVYVHKINKVDDVVQYIEKHQTQTISFYPWESSKKYRDAFAAKGVERIVESGMNNIFRAGGAHDAMRPLQRLVRFVSHERPYNFTTKDVSVEIEQTRFLEEDKFLVFVP

SEQ ID NO: 29 - protein, 479 aa, Vibrio fischeri
MIKCIPMIIKGVVQDFDNNACKEINLDSGNKIKLSLLTEDSVLRSLNSKEKVDLNLNQIVNFLYTVGQRWKNEEYNRRRTYIRELKKYLGYSDEMARLEANWIAMLLCSKSALYDIVNYDLGSIHVLDEWLPRGDCYVKAQAKGVSIHLLAGNVPLSGVTSILRAILTKNECIIKTSSSDPFTATALASSFIDVNAEHPITKSMSVMYWPHNEDMTLPQRIMNHADIVIAWGGEEAIKWAAKHSPPHADVLKFGPKKSLSIIEEPEDMEEAAMGVAHDICFYDQQACFSTQDVYYIGEHLPLFLSELEKQLDRYAKILPKGLKNFDEKAAFSLTEREGIFAGYDVKKGDNQAWLMIISPTNSSGNQPLSRSVYIHQVSDINEVLPFVNKNSTQTVSIYPWEASLKYRDKLAMSGAERIVESGMNNIFRVGGAHDSLSPLQYLVRFTSHERPFHYTTKDVAVEIEQTRYLEEDKFLVFVP

SEQ ID NO: 30 - protein, 305 aa, Vibrio harveyi
MNNQCKTIAHVLRVNNGQELHVWETPPKENVPSKNNTILIASGFARRMDHFAGLAEYLSENGFHVFRYDSLHHVGLSSGSIDEFTMTTGKNSLCTVYHWLQTKGTQNIGLIAASLSARVAYEVISDLELSFLITAVGVVNLRDTLEKALGFDYLSLPIDELPNDLDFEGHKLGSEVFVRDCFEHHWDTLDSTLDKVANTSVPLIAFTANNDDWVKQEEVYDMLAHIRTGHCKLYSLLGSSHDLGENLVVLRNFYQSVTKAAIAMDGGSLEIDVDFIEPDFEQLTIATVNERRLKAEIESRTPEMA

SEQ ID NO: 31 - protein, 307 aa, Vibrio fischeri
MKDESALFTIDHIIKLDNGQSIRVWETLPKKNVPEKKNTILIASGFARRMDHFAGLAEYLSTNGFHVIRYDSLHHVGLSSGCINEFTMSIGKNSLLTVVDWLTDHGVERIGLIAASLSARIAYEVVNKIKLSFLITAVGVVNLRDTLEKALEYDYLQLPISELPEDLDFEGHNLGSEVFVTDCFKHDWDTLDSTLNSVKGLAIPFIAFTANDDSWVKQSEVIELIDSIESSNCKLYSLIGSSHDLGENLVVLRNFYQSVTKAALALDDGLLDLEIDIIEPRFEDVTSITVKERRLKNEIENELLELA

SEQ ID NO: 32 - protein, 378 aa, Vibrio harveyi
MDVLSAVKQENIAASTEIDDLIFMGTPQQWSLQEQKQLTSRLVKGAYQYHYHNNDDYRQFCERLGVGEVVEDLNDIPVFPTSIFKLKTLLTLDDDEVENRFTSSGTSGIKSIVARDRLSIERLLGSVNFGMNYVGDWFDHQMELVNLGPDRFNANNIWFKYVMSLVELLYPTAFTVTEDEIDFEATLANMNRIKQSGKTICLIGPPYFIYLLCCFMREQGQTFNGGRDLYIITGGGWKKHQDQSLDRDEFNQLLCETFTLESPEQIRDTFNQVELNTCFFEDTEHKKRVPPWVFARALDPKTLKPLPHGQPGLMSYMDASAVSYPCFLVTDDIGIVREEEGDRPGTTVEIVRRVKTRGMKGCALSMSQAFTAKSEGGN

SEQ ID NO: 33 - protein, 376 aa, Vibrio fischeri
MTNHIEYKKNQIIASSEIDDLIFMSAPQEWSLEEQKEIQDKLVREAFHFHYNRNEKYRNYCISQHINENLHSIDEIPVFPTSIFKHMKFHTVSMGDIENWHTSSGTQGIKSCIARDRLSIERLLGSVNFGMKYVGNWFEHQMELVNLGPDRFSASNVWFKYVMSLVELLYPTVFTVNNDKIDFEETVNHLYRINNSNKDICLIGPPFFVSLLCQYMKENNIEFKGENRLHVITGGGWKSNENSSLNRQDFNQLIMDTFQLDNVNQIRDTFNQVELNTCFFEDEFQRKHVPPWVYARALDPETLKPVADGELGLLSYMDASSTAYPAFIVTDDIGIVREIREPDPYPGVTVEIVRRLNTRAQKGCALSMASFIQSTI